SEQUENCE LISTING (2 sequences)

SEQ ID NO: 1 - DNA, 11 bp, synthetic construct
  source 1..11: mol_type = other DNA
ggaagaatcac

SEQ ID NO: 2 - DNA, 11 bp, synthetic construct
  source 1..11: mol_type = other DNA
ggaagaatcac

(19) United States
(12) Patent Application Publication
(10) Pub. No.: US 2026/0209766 A1
(43) Pub. Date: Jul. 23, 2026

(54) CRIPPLED CORONAVIRUS: 5'-POLYU TARGETED OLIGO PREVENTS DEVELOPMENT OF INFECTIOUS VIRIONS

(71) Applicant: Howard University, Washington, DC (US)

(72) Inventors: Hemayet ULLAH, Washington, DC (US); Saadyah AVERICK, Dallas, TX (US)

(73) Assignee: Howard University, Washington, DC (US)

(21) Appl. No.: 19/157,806

(22) PCT Filed: Feb. 21, 2024
(86) PCT No.: PCT/US2024/016650

§ 371 Date: Aug. 19, 2025

Related U.S. Application Data
(60) Provisional application No. 63/486,140, filed on Feb. 21, 2023.

(51) Int. Cl. C12N 15/113; A61K 9/51; A61K 31/712; A61K 31/713; A61P 31/04

(52) U.S. Cl. CPC C12N 15/1131; A61K 9/5138; A61K 9/5146; A61K 31/712; A61K 31/713; A61P 31/04; C12N 2310/14; C12N 2310/321

(57) ABSTRACT

The present disclosure provides compositions and methods for treating and detecting coronavirus infection, and in particular, provides compositions and methods for treating and detecting SARS-CoV-2 infection. The present disclosure also relates to the production of compositions for treating and detecting coronavirus infection, and in particular, to the production of compositions for treating and detecting SARS-CoV-2 infection.

### STATEMENT REGARDING FEDERALLY SPONSORED RESEARCH OR DEVELOPMENT

Not applicable.

### SEQUENCE LISTING

The present application contains a Sequence Listing that is hereby incorporated by reference in its entirety.

### TECHNICAL FIELD

The present disclosure relates generally to compositions and methods for treating and detecting virus infection, and in particular, relates to compositions and methods for treating and detecting coronavirus infection, including Severe acute respiratory syndrome coronavirus 2 (SARS-CoV-2) infection. The disclosure also relates to the production of compositions for treating and detecting virus infection, and in particular, to the production of compositions for treating and detecting coronavirus infection, including SARS-CoV-2 infection.

### BACKGROUND

As of 2022, SARS-CoV-2 has infected more than six-hundred million individuals worldwide, resulting in more than 6 million deaths to date. Although the development of vaccines against SARS-CoV-2 has been rapid, there remain no effective treatments against the virus. Hence, for individuals who have contracted SARS-CoV-2; or who are unable to be vaccinated because of underlying health conditions or personal reasons, for example, there remains an urgent need for an effective treatment that is able to inhibit proliferation of the virus.

Additionally, because of the intrinsic ability of SARS-CoV-2 to accumulate genomic mutations at a high rate, it is conceivable that future SARS-CoV-2 variants may be resistant to existing vaccines, or vaccines in the late stages of development. Concerns surround whether drug-escape-variants, like immune-escape-variants, can evolve against any drug developed targeting the virus.

At present, individuals infected with SARS-CoV-2 generally receive supportive care, such as supplemental oxygen, mechanical ventilation, and steroids. None of these supportive therapies target or inhibit SARS-CoV-2, and their success ultimately relies upon the host's immune system to control the virus. A therapy specifically targeted at SARS-CoV-2 is therefore desirable.

However, the identification of a suitable drug target for SARS-CoV-2, much less the identification of a drug that specifically binds that target, is not straightforward; because existing antiviral drugs (such as nucleoside analogs, for example) often exhibit significant side effects against the host, their use can be problematic. Additionally, viral mismatch repair mechanisms may limit the analogs' effectiveness.

Accordingly, the development of a treatment that targets SARS-CoV-2 and/or inhibits its interaction with the host cell machinery, but which shows little or no serious side effects against the host, is especially desirable.

Since targeting coding regions directly can potentially lead to the evolution of escape variants, mitigation strategies should consider a mechanism to prevent the development of any escape-variants. One effective targeted strategy is to block an area of the virus genome that is absolutely required by the virus to complete its life cycle.

The 5′ polyuridines (5′ poly-U) tract on the antigenome (minus RNA genome) of the positive RNA virus is indispensable for the virus as it is used as a template to generate the 3′ poly-A tail in its +RNA genome and the 3′ poly-A tail serves as template to generate the 5′ poly-U tract on the antigenome.

Any mutation within the 5′ poly-U tract will most likely prevent the virus from generating its 3′ poly-A tail, which would effectively hamper the generation of its full-length genome to complete its life cycle. The essentiality of the homopolymorphic 5′ poly-U tract on the antigenome for the survival and infectivity of positive RNA viruses, including many coronaviruses, is well-established. On the other hand, host cells do not harbor any 5′ poly-U tracts on any of their transcripts, making the tract an attractive, virus-specific target. Eukaryotic RNA polymerase III transcribes some small RNAs (t-RNA and 5S rRNA) with 3′ poly-U tract as a part of the transcription termination mechanism. Molecular distinction between the viruses' 5′ poly-U tract from the host's 3′ poly-U tract on transcripts will be a central consideration to avoid any off-target effects.

Targeting the 5′ poly-U of a coronavirus antigenome, by providing a complimentary 3′ poly-dA-containing oligo with an additional 5′-end linker region that comprises a sequence complementary, or nearly complementary, to a sequence at the 5′-end of the negative-strand RNA that starts immediately after the 5′ poly-U stretch to confer specificity, is capable of protecting infected cells from virus-induced cytopathic effects. It also diminishes the ability of the virus to escape the host cell and infect neighboring cells.

There is nothing in the art that suggests using a poly-A antisense oligonucleotide that targets the coronavirus antigenome, as well as potentially introducing a frameshift mutation into the coronavirus genome.

### SUMMARY OF THE INVENTION

The present disclosure provides novel viral and host protein targets that, when inhibited in combination, can inhibit viral proliferation synergistically. In particular, the viral targets are ribonucleic acid sequences present in, or generated by, coronaviruses, including SARS-CoV-2, while the host protein targets are proteins utilized by the coronaviruses for proliferation.

In embodiments herein, the novel viral targets are present in a region that is not amenable to mutation (e.g., because mutations in the region would disrupt the viral replication cycle). In certain embodiments, the viral target is a 5′ poly-uracil (“5′ poly-U”) sequence present in a negative-sense RNA molecule transcribed from a coronavirus positive-sense viral genome, such as the SARS-CoV-2 positive-sense viral genome. Because the 5′ poly-U is indispensable for the virus, any drugs developed targeting the 5′ poly-U will be durable (virus will not be able to evade the drugs by mutating).

In embodiments herein, the effect of inhibiting the novel viral targets that are present in a region that is not amenable to mutation is further enhanced by using oligos that could introduce mutations into the coronavirus genome.

The present disclosure further provides nucleic acid molecules, and compositions containing such nucleic acid molecules, that hybridize to the 5′ poly-U sequence in a negative-sense RNA molecule transcribed from a coronavirus positive-sense viral genome, such as the SARS-CoV-2 positive-sense viral genome.

Non-limiting embodiments of the disclosure include as follows.

[1] A composition comprising; a first agent that binds to a negative-strand ribonucleic acid (RNA) molecule produced by a coronavirus, wherein the negative-strand ribonucleic acid (RNA) molecule comprises a 5′ poly-U stretch, and wherein the first agent is selected from the group consisting of a nucleic acid, an antibody, a protein, an aptamer, and a low molecular weight compound.

[2] The composition of [1] wherein the first agent is a nucleic acid molecule.

[3] The composition of [2] wherein the nucleic acid molecule comprises a poly-A stretch that is between 5-50 bases in length, and wherein the poly-A stretch is capable of hybridizing to the 5′ poly-U stretch in the negative-strand RNA molecule.

[4] The composition of [3] wherein the nucleic acid molecule further comprises a 5′-end linker region that comprises a sequence complementary, or nearly complementary, to a sequence at the 5′-end of the negative-strand RNA that starts immediately after the poly-U stretch.

[5] The composition of [4] wherein the linker sequence in the nucleic acid molecule comprises GGAAGAATCAC (nucleotides 1-11 of SEQ ID NO: 1).

[6] The composition of [4] wherein the linker sequence in the nucleic acid molecule comprises GAATTGGAAGATCAC (nucleotides 1-15 of SEQ ID NO: 2).

[7] The composition of [2] wherein the nucleic acid molecule contains at least one of a nucleic acid analogue, a base analog, a modified base, and an abasic site.

[8] The composition of [7] wherein the at least one of a nucleic acid analogue, a base analog, a modified base, and an abasic site is the 3′ terminal nucleic acid.

[9] The composition of [7] wherein the at least one of a nucleic acid analogue, a base analog, a modified base, and an abasic site is the 5′ terminal nucleic acid.

[10] The composition of [7] wherein the at least one of a nucleic acid analogue, a base analog, a modified base, and an abasic site is both the 3′ and 5′ terminal nucleic acids.

[11] The composition of [10] wherein the 3′ and 5′ terminal nucleic acid modifications are the same.

[12] The composition of [10] wherein the 3′ and 5′ terminal nucleic acid modifications are different.

[13] The composition of [7] wherein at least one of the nucleic acid analogues is a 2′MOE ribonucleic acid.

[14] The composition of [2] wherein the nucleic acid molecule is conjugated to a heterologous molecule.

[15] The composition of [14] wherein the heterologous molecule is a detectable label.

[16] The composition of [14] wherein the heterologous molecule is a molecule that targets the nucleic acid molecule to a cell or intracellular compartment thereof.

[17] The composition of [4] wherein the nucleic acid molecule comprises a nucleotide sequence selected from the group consisting of SEQ ID NOs: 1-2.

[18] The composition of [1] wherein the composition further comprises a delivery vehicle.

[19] The composition of [18] wherein the delivery vehicle is a polymer.

[20] The composition of [19] wherein the polymer is selected from the group consisting of a polyethylenimine polymer and a linear poly methacrylate cationic polymer.

[21] The composition of [20] wherein the polyethylenimine polymer is a branched polyethylenimine polymer.

[22] The composition of [18] wherein the delivery vehicle is selected from the group consisting of a lipid, a liposome, a hydrogels, a cyclodextrin, poly(lactic-co-glycolic)acid (PLGA), a microsphere, a nanocapsule, and a protein.

[23] The composition of [1] wherein the virus is from the family Coronaviridae.

[24] The composition of [23] wherein the virus is MHV.

[25] The composition of [23] wherein the virus is SARS-CoV-2.

[26]A method for treating a coronavirus infection, comprising administering to a subject a composition comprising; a first agent that binds to a negative-strand ribonucleic acid (RNA) molecule produced by a coronavirus, wherein the negative-strand ribonucleic acid (RNA) molecule comprises a 5′ poly-U stretch, and wherein the first agent is selected from the group consisting of a nucleic acid, an antibody, a protein, an aptamer, and a low molecular weight compound.

[27] The method of [26], wherein the a coronavirus infection is a SARS-CoV-2 infection.

[28] The method of [26], wherein the a coronavirus is SARS-CoV-2.

[29] The method of [26], wherein the first agent is a nucleic acid molecule.

[30] The method of [29], wherein the nucleic acid molecule comprises a poly-A stretch that is between 5-50 bases in length, and wherein the poly-A stretch is capable of hybridizing to the 5′ poly-U stretch in the negative-strand RNA molecule.

[31] The method of [30] wherein the nucleic acid molecule further comprises a 5′-end linker region that comprises a sequence complementary, or nearly complementary, to a sequence at the 5′-end of the negative-strand RNA that starts immediately after the poly-U stretch.

[32] The method of [31] wherein the linker sequence in the nucleic acid molecule comprises GGAAGAATCAC (nucleotides 1-11 of SEQ ID NO: 1).

[33] The nucleic acid molecule of [31] wherein the linker sequence in the nucleic acid molecule comprises GAATTGGAAGATCAC (nucleotides 1-15 of SEQ ID NO: 2).

[34] The method of [29] wherein the nucleic acid molecule contains at least one of a nucleic acid analogue, a base analog, a modified base, and an abasic site.

[35] The method of [34] wherein the at least one of a nucleic acid analogue, a base analog, a modified base, and an abasic site is the 3′ terminal nucleic acid.

[36] The method of [34] wherein the at least one of a nucleic acid analogue, a base analog, a modified base, and an abasic site is the 5′ terminal nucleic acid.

[37] The method of [34] wherein the at least one of a nucleic acid analogue, a base analog, a modified base, and an abasic site is both the 3′ and 5′ terminal nucleic acids.

[38] The method of [37] wherein the 3′ and 5′ terminal nucleic acid modifications are the same.

[39] The method [37] wherein the 3′ and 5′ terminal nucleic acid modifications are different.

[40] The method [34] wherein at least one of the nucleic acid analogues is a 2′MOE ribonucleic acid.

[41] The method [29] wherein the nucleic acid molecule is conjugated to a heterologous molecule.

[42] The method [41] wherein the heterologous molecule is a detectable label.

[43] The method [41] wherein the heterologous molecule is a molecule that targets the nucleic acid molecule to a cell or intracellular compartment thereof.

[44] The method [29] wherein the nucleic acid molecule comprises a nucleotide sequence selected from the group consisting of SEQ ID NOs: 1-2.

[45] The method [26] wherein the composition further comprises a delivery vehicle.

[46] The method [45] wherein the delivery vehicle is a polymer.

[47] The method [46] wherein the polymer is selected from the group consisting of a polyethylenimine polymer and a linear poly methacrylate cationic polymer.

[48] The method [47] wherein the polyethylenimine polymer is a branched polyethylenimine polymer.

[49] The method [45] wherein the delivery vehicle is selected from the group consisting of a lipid, a liposome, a hydrogels, a cyclodextrin, poly(lactic-co-glycolic)acid (PLGA), a microsphere, a nanocapsule, and a protein.

[50] The method [26] wherein the virus is from the family Coronaviridae.

[51] The method [50] wherein the virus is MHV.

[52] The method [50] wherein the virus is SARS-CoV-2.

### INCORPORATION BY REFERENCE

All patents, publications, and patent applications cited in the present specification are herein incorporated by reference as if each individual patent, publication, or patent application was specifically and individually indicated to be incorporated by reference in its entirety for all purposes.

### BRIEF DESCRIPTION OF THE FIGURES

FIG. 1A. Illustration of the organization of the SARS-CoV-2 genome.

FIG. 1B. Illustration of the organization of the Murine Hepatitis Virus genome.

FIG. 2A. Oligo sequences designed to be complementary to the 5′ end of the minus strand of Murine hepatitis virus strain MHV-A59 genome. The dash shows the one base deletion from the 3′UTR sequence of the Bison Oligo-2, while the corresponding base in Bison Oligo-1 is underlined.

FIG. 2B. Structures of the 5′ (top panel) and 3′ (bottom panel) modifications of the sequences in FIG. 2A.

FIG. 2C. Molecular structures of the block-copolymer (Polymer 1 and Polymer 2), and the branched polyethylenimine (bPEI) (Polymer 3), used to deliver the oligonucleotides to the host cell.

FIG. 3. Treatment with Bison Oligo-1 or Bison Oligo-2 prevents MHV-A59-eGFP induced infection in fluorescently tagged secondary 17CL-1 cells (magenta colored).

FIG. 3A. Negative control with only MHV-A59-eGFP and Polymer1. Arrow indicates infection induced syncytium (Original magnification 10×). Scale bar 100 μm. FIG. 3B. MHV-A59-eGFP with Polymer1 and Bison Oligo-1. FIG. 3C. MHV-A59-eGFP with Polymer1 and Bison Oligo-2.

FIG. 4. Detection of MHV-A59 nucleoprotein N in released virions in the media (Panel A) or virions inside cell (Panel B). Respective treatments are shown on the top of each gel.

FIG. 5. Infectivity assay done with the media released virions. Lower panel shows image after 6 h in bright field. Upper panel shows image after 24 h in bright field.

FIG. 6. Immunofluorescence visualization of dsRNA during MHV-A59-eGFP active infection of 17CL-1 cells in the absence (Panel A) or in the presence of Bison Oligo-1 (Panel B) or in the presence of oligonucleotide 2 (Panel C).

FIG. 7. Bison Oligo-1 targeting the MHV 5′ polyU on minus RNA strand prevented MHV induced syncytium formation in mouse 17CL-1 cells. Syncytium identified with arrows.

FIG. 8. Bison Oligo-1 targeting the MHV 5′ polyU on minus RNA strand prevented MHV induced inhibition of cell cycle Marker-CDC25A.

FIG. 9. Image-based flowcytometry shows oligo induced rescue of cell cycle arrest caused by infection with MHV.

### DETAILED DESCRIPTION OF THE INVENTION

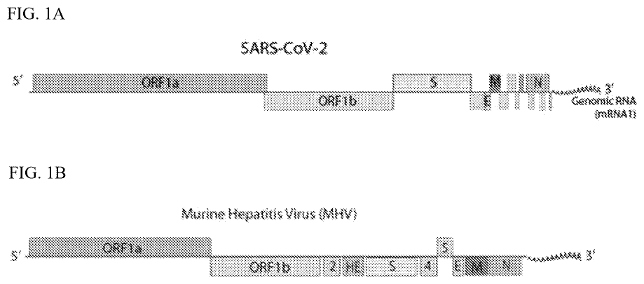

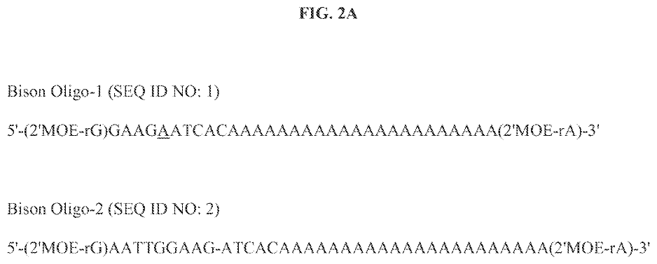

SARS-CoV-2, the causative agent of COVID-19, is an enveloped RNA virus within the family Coronaviridae, and contains a single-stranded, positive-sense (+), RNA genome. The organization of the SARS-CoV-2 genome is depicted in FIG. 1. This is in contrast to the human genome, which is double-stranded DNA. Infection of a cell with the SARS-CoV-2 virus begins with attachment of the SARS-CoV-2 spike (S) protein to its cognate cellular receptor (angiotensin-converting enzyme 2; ACE2), followed by internalization, fusion with the endosomal membrane and uncoating, and translation of viral non-structural proteins (encoded by the single-stranded, positive-sense (+), RNA genome of SARS-CoV-2) to form the viral replicase complex. Following assembly of viral replicase complexes, viral RNA synthesis ensues, which results in the production of both genomic and sub-genomic RNAs (the sub-genomic RNAs are used as mRNAs for the translation of structural and accessory genes). The sub-genomic mRNA cannot serve as templates for negative-strand synthesis, because they lack the 5′-replication signal.

$$Pos sense = mRNA ( Covid )$$

$$Neg sense = template for mRNA$$

The genomic and sub-genomic RNAs are produced using negative-strand intermediates that contain, amongst others, 5′ poly-Uridine (poly-U) sequences complementary to the polyadenylated tail of the single-stranded, positive-sense (+), RNA genome of SARS-CoV-2. Similar to eukaryotic mRNA, the SARS-CoV-2 genome is both 5′-capped, and 3′ polyadenylated (in a template-dependent fashion).

The negative-strand intermediates are synthesized throughout infection (via RNA-dependent RNA polymerase), although their synthesis declines after about six- or seven hours post-infection. In contrast, human cells do not typically produce any negative-strand RNA molecules, and therefore, 5′ poly-U-containing nucleic acids are typically absent from normal human cells (although some small RNA transcripts synthesized by host RNA polymerase III contain a short stretch of poly-U at only the 3′-end).

As described herein, the present inventors have demonstrated that targeting the 5′ poly-U tract of the negative-strand intermediates inhibits SARS-CoV-2 viral replication (blocking the poly-U tract prevents genomic and sub-genomic mRNA synthesis). Moreover, because 5′ poly-U-containing nucleic acids are typically absent from normal human cells, blocking the 5′ poly-U tract of the viral negative-strand intermediates is not associated with off-target effects.

To further enhance targeting and specificity, and to further reduce off-target effects, the 5′ poly-U sequences may be targeted using a polynucleotide that comprises a poly-A sequence and which further comprises a 5′-end linker region that comprises a sequence complementary to a sequence at the 5′-end of the negative-strand viral RNA that starts immediately after the poly-U stretch. In this configuration, both the poly-A sequence and the 5′-end linker region sequence will hybridize to the SARS-CoV-2 negative-strand intermediates.

It is to be understood that the terminology used herein is for the purpose of describing particular embodiments only and is not intended to be limiting. As used in the present specification and the appended claims, the singular forms “a,” “an,” and “the” include plural referents unless the context clearly dictates otherwise. Thus, for example, reference to “a polynucleotide” includes one or more polynucleotides, and reference to “a vector” includes one or more vectors.

Unless defined otherwise, all technical and scientific terms used herein have the same meaning as commonly understood by one of ordinary skill in the art to which the invention pertains. Although other methods and materials similar, or equivalent, to those described herein can be useful in the present invention, preferred materials and methods are described herein.

In view of the teachings of the present specification, one of ordinary skill in the art can apply conventional techniques of immunology, biochemistry, chemistry, molecular biology, microbiology, cell biology, genomics, and recombinant polynucleotides, as taught, for example, by the following standard texts: Abbas et al. (Cellular and Molecular Immunology, 2017, 9th Edition, Elsevier, ISBN 978-0323479783); Butterfield et al. (Cancer Immunotherapy Principles and Practice, 2017, 1st Edition, Demos Medical, ISBN 978-1620700976); Kenneth Murphy (Janeway's Immunobiology, 2016, 9th Edition, Garland Science, ISBN 978-0815345053); Stevens et al. (Clinical Immunology and Serology: A Laboratory Perspective, 2016, 4th Edition, Davis Company, ISBN 978-0803644663); E. A. Greenfield (Antibodies: A Laboratory Manual, 2014, Second edition, Cold Spring Harbor Laboratory Press, ISBN 978-1-936113-81-1); R. I. Freshney (Culture of Animal Cells: A Manual of Basic Technique and Specialized Applications, 2016, 7th Edition, Wiley-Blackwell, ISBN 978-1118873656); C. A. Pinkert (Transgenic Animal Technology, Third Edition: A Laboratory Handbook, 2014, Elsevier, ISBN 978-0124104907); H. Hedrich (The Laboratory Mouse, 2012, Second Edition, Academic Press, ISBN 978-0123820082); Behringer et al. (Manipulating the Mouse Embryo: A Laboratory Manual, 2013, Fourth Edition, Cold Spring Harbor Laboratory Press, ISBN 978-1936113019); McPherson et al. (PCR 2: A Practical Approach, 1995, IRL Press, ISBN 978-0199634248); J. M. Walker (Methods in Molecular Biology (Series), Humana Press, ISSN 1064-3745); Rio et al. (RNA: A Laboratory Manual, 2010, Cold Spring Harbor Laboratory Press, ISBN 978-0879698911); Methods in Enzymology (Series), Academic Press; Green et al. (Molecular Cloning: A Laboratory Manual, 2012, Fourth Edition, Cold Spring Harbor Laboratory Press, ISBN 978-1605500560); and G. T. Hermanson (Bioconjugate Techniques, 2013, Third Edition, Academic Press, ISBN 978-0123822390).

A “linker region sequence,” “linker sequence,” and “linker polynucleotide” are used interchangeably herein and refer to a sequence of one or more nucleotides covalently attached to a first nucleic acid sequence (e.g., 5′-linker nucleotide sequence-first nucleic acid sequence-3′). In some embodiments, a linker nucleotide sequence connects two separate nucleic acid sequences to form a single polynucleotide (e.g., 5′-first nucleic acid sequence-linker nucleotide sequence-second nucleic acid sequence-3′). Other examples of linker sequences include, but are not limited to, 5-first nucleic acid sequence-linker nucleotide sequence-3′, and 5′-linker nucleotide sequence-first first nucleic acid sequence-linker nucleotide sequence-3′. In some embodiments, the linker nucleotide sequence can be a single-stranded nucleotide sequence of unpaired nucleic acid bases that do not interact with each other through hydrogen bond formation to create a secondary structure. In some embodiments, a linker element nucleotide sequence can be about 100 or less, about 90 or less, about 80 or less, about 70 or less, about 60 or less, about 50 or less, about 40 or less, about 30 or less, about 20 or less, about 15 or less, about 14 or less, about 13 or less, about 12 or less, about 11 or less, about 10 or less, about 9 or less, about 8 or less, about 7 or less, about 6 or less, about 5 or less, about 4 or less, about 3 or less, or about 2 or less, bases in length.

The terms “wild-type,” “naturally occurring,” and “unmodified” are used herein to mean the typical (or most common) form, appearance, phenotype, or strain existing in nature; for example, the typical form of cells, organisms, polynucleotides, proteins, macromolecular complexes, genes, RNAs, DNAs, or genomes as they occur in, and can be isolated from, a source in nature. The wild-type form, appearance, phenotype, or strain serve as the original parent before an intentional modification. Thus, mutant, variant, engineered, recombinant, and modified forms are not wild-type forms.

By “isolated” is meant, when referring to a polypeptide, that the indicated molecule is separate and discrete from the whole organism with which the molecule is found in nature or is present in the substantial absence of other biological macromolecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The term “purified” as used herein preferably means at least 75% by weight, more preferably at least 85% by weight, more preferably still at least 95% by weight, and most preferably at least 98% by weight, of the same molecule is present.

The terms “engineered,” “genetically engineered,” “genetically modified,” “recombinant,” “modified,” “non-naturally occurring,” and “non-native” indicate intentional human manipulation of the genome of an organism or cell. The terms encompass methods of genomic modification that include genomic editing, as defined herein, as well as techniques that alter gene expression or inactivation, enzyme engineering, directed evolution, knowledge-based design, random mutagenesis methods, gene shuffling, codon optimization, and the like. Methods for genetic engineering are known in the art.

“Oligo1,” “Oligonucleotide 1” and “Bison Oligo-1” are used interchangeably herein. “Oligo2,” “Oligonucleotide 2” and “Bison Oligo-2” are also used interchangeably herein.

“Covalent bond,” “covalently attached,” “covalently bound,” “covalently linked,” “covalently connected,” and “molecular bond” are used interchangeably herein and refer to a chemical bond that involves the sharing of electron pairs between atoms. Examples of covalent bonds include, but are not limited to, phosphodiester bonds and phosphorothioate bonds.

“Non-covalent bond,” “non-covalently attached,” “non-covalently bound,” “non-covalently linked,” “non-covalent interaction,” and “non-covalently connected” are used interchangeably herein, and refer to any relatively weak chemical bond that does not involve sharing of a pair of electrons. Multiple non-covalent bonds often stabilize the conformation of macromolecules and mediate specific interactions between molecules. Examples of non-covalent bonds include, but are not limited to hydrogen bonding, ionic interactions (e.g., Na⁺Cl⁻), van der Waals interactions, and hydrophobic bonds.

As used herein, “hydrogen bonding,” “hydrogen-base pairing,” and “hydrogen bonded” are used interchangeably and refer to canonical hydrogen bonding and non-canonical hydrogen bonding including, but not limited to, “Watson-Crick-hydrogen-bonded base pairs” (W-C-hydrogen-bonded base pairs or W-C hydrogen bonding); “Hoogsteen-hydrogen-bonded base pairs” (Hoogsteen hydrogen bonding); and “wobble-hydrogen-bonded base pairs” (wobble hydrogen bonding).

W-C hydrogen bonding, including reverse W-C hydrogen bonding, refers to purine-pyrimidine base pairing, that is, adenine:thymine, guanine:cytosine, and uracil:adenine. Hoogsteen hydrogen bonding, including reverse Hoogsteen hydrogen bonding, refers to a variation of base pairing in nucleic acids wherein two nucleobases, one on each strand, are held together by hydrogen bonds in the major groove. This non-W-C hydrogen bonding can allow a third strand to wind around a duplex and form triple-stranded helices. Wobble hydrogen bonding, including reverse wobble hydrogen bonding, refers to a pairing between two nucleotides in RNA molecules that does not follow Watson-Crick base pair rules. There are four major wobble base pairs: guanine:uracil, inosine (hypoxanthine):uracil, inosine:adenine, and inosine:cytosine. Wobble base interaction are also known to occur between inosine:thymine and inosine:guanine. Inosine bases and deoxy inosine bases can be referred to as “universal pairing bases” as they are capable of hydrogen bonding with the canonical DNA and RNA bases (see, e.g., FIG. 7). See also Watkins et al. (Nucleic Acid Research, 2005, 33(19):6258-67). Rules for canonical hydrogen bonding and non-canonical hydrogen bonding are known to those of ordinary skill in the art. See, e.g., R. F. Gesteland (The RNA World, Third Edition (Cold Spring Harbor Monograph Series), 2005, Cold Spring Harbor Laboratory Press, ISBN 978-0879697396); R. F. Gesteland (The RNA World, Second Edition (Cold Spring Harbor Monograph Series), 1999, Cold Spring Harbor Laboratory Press, ISBN 978-0879695613); R. F. Gesteland (The RNA World, First Edition (Cold Spring Harbor Monograph Series), 1993, Cold Spring Harbor Laboratory Press, 978-0879694562) (see, e.g., Appendix 1: Structures of Base Pairs Involving at Least Two Hydrogen Bonds, I. Tinoco); W. Saenger (Principles of Nucleic Acid Structure, 1988, Springer International Publishing AG, ISBN 978-0-387-90761-1); S. Neidle (Principles of Nucleic Acid Structure, 2007, First Edition, Academic Press, ISBN 978-01236950791).

“Connect,” “connected,” and “connecting” are used interchangeably herein, and refer to a covalent bond or a non-covalent bond between two macromolecules (e.g., polynucleotides, proteins, and the like).

As used herein, the terms “nucleic acid sequence,” “nucleotide sequence,” and “oligonucleotide” are interchangeable and refer to a polymeric form of nucleotides. As used herein, the term “polynucleotide” refers to a polymeric form of nucleotides that has one 5′ end and one 3′ end and can comprise one or more nucleic acid sequences. The nucleotides may be deoxyribonucleotides (DNA), ribonucleotides (RNA), analogs thereof, or combinations thereof, and may be of any length. Polynucleotides may perform any function and may have various secondary and tertiary structures. The terms encompass known analogs of natural nucleotides and nucleotides that are modified in the base, sugar, and/or phosphate moieties. Analogs of a particular nucleotide have the same base-pairing specificity (e.g., an analog of A base pairs with T). A polynucleotide may comprise one modified nucleotide or multiple modified nucleotides. Examples of modified nucleotides include fluorinated nucleotides, methylated nucleotides, and nucleotide analogs. Nucleotide structure may be modified before or after a polymer is assembled. Following polymerization, polynucleotides may be additionally modified via, for example, conjugation with a labeling component or target binding component. A nucleotide sequence may incorporate non-nucleotide components. The terms also encompass nucleic acids comprising modified backbone residues or linkages, that are synthetic, naturally occurring, and/or non-naturally occurring, and have similar binding properties as a reference polynucleotide (e.g., DNA or RNA). Examples of such analogs include, but are not limited to, phosphorothioates, phosphoramidates, methyl phosphonates, chiral-methyl phosphonates, 2-O-methyl ribonucleotides, peptide-nucleic acids (PNAs), Locked Nucleic Acid (LNA™) (Exiqon, Woburn, MA) nucleosides, glycol nucleic acid, bridged nucleic acids, and morpholino structures.

As used herein, the term “poly-U” refers to a plurality of continuous uracil bases. Similarly, the term “poly-A” refers to a plurality of continuous adenine bases. It is understood that a continuous stretch of poly-U or poly-A, for example, may contain some modified bases, base analogs, or abasic sites, and still be considered a poly-U or poly-A, respectively.

As used herein, the terms “abasic,” “abasic site,” “abasic nucleotide,” and “apurinic/apyrimidinic site,” are used interchangeably and refer to a site in a nucleotide sequence that lacks the purine or a pyrimidine base. In certain embodiments, abasic sites comprise a deoxyribose site. In other embodiments, abasic sites comprise a ribose site. In yet further embodiments, abasic sites comprise a modified backbone, such as a pentose ring with a 1′ hydroxyl group. An abasic site cannot form hydrogen base pair bonding with a complementary nitrogen base of a DNA or RNA nucleotide, because it does not contain a nitrogen base.

As used herein, the term “base analog” refers to a compound having structural similarity to a canonical purine or pyrimidine base occurring in DNA or RNA. The base analog may contain a modified sugar and/or a modified nucleobase, as compared to a purine or pyrimidine base occurring naturally in DNA or RNA. In some embodiments, the base analog is inosine or deoxyinosine, such as 2′-deoxyinosine. In other embodiments, the base analog is a 2′-deoxyribonucleoside, 2′-ribonucleoside, 2′-deoxyribonucleotide or a 2′-ribonucleotide, wherein the nucleobase includes a modified base (such as, for example, xanthine, uridine, oxanine (oxanosine), 7-methlguanosine, dihydrouridine, 5-methylcytidine, C3 spacer, 5-methyl dC, 5-hydroxybutynl-2′-deoxyuridine, 5-nitroindole, 5-methyl iso-deoxycytosine, iso deoxyguanosine, deoxyuradine, iso deoxycytidine, other 0-1 purine analogs, N-6-hydroxylaminopurine, nebularine, 7-deaza hypoxanthine, other 7-deazapurines, and 2-methyl purines). In some embodiments, the base analog may be selected from the group consisting of 7-deaza-2′-deoxyinosine, 2′-aza-2′-deoxyinosine, PNA-inosine, morpholino-inosine, LNA-inosine, phosphoramidate-inosine, 2′-O-methoxyethyl-inosine, and 2′-OMe-inosine. The term “base analog” also includes, for example, 2′-deoxyribonucleosides, 2′-ribonucleosides, 2′-deoxyribonucleotides or 2′-ribonucleotides, wherein the nucleobase is a substituted hypoxanthine. For instance, the substituted hypoxanthine may be substituted with a halogen, such as fluorine or chlorine. In some embodiments, the base analog may be a fluoroinosine or a chloroinosine, such as 2-chloroinosine, 6-chloroinosine, 8-chloroinosine, 2-fluoroinosine, 6-fluoroinosine, or 8-fluoroinosine. In other embodiments, the base analog is deoxyuradine. In other embodiments the base analog is a nucleic acid mimic (such as, for example, artificial nucleic acids and xeno nucleic acids (XNA).

Peptide-nucleic acids (PNAs) are synthetic homologs of nucleic acids wherein the polynucleotide phosphate-sugar backbone is replaced by a flexible pseudo-peptide polymer. Nucleobases are linked to the polymer. PNAs have the capacity to hybridize with high affinity and specificity to complementary sequences of RNA and DNA.

In phosphorothioate nucleic acids, the phosphorothioate (PS) bond substitutes a sulfur atom for a non-bridging oxygen in the polynucleotide phosphate backbone. This modification makes the internucleotide linkage resistant to nuclease degradation. In some embodiments, phosphorothioate bonds are introduced between the last 3 to 5 nucleotides at the 5′-end or 3′-end sequences of a polynucleotide sequence to inhibit exonuclease degradation. Placement of phosphorothioate bonds throughout an entire oligonucleotide helps reduce degradation by endonucleases as well.

Threose nucleic acid (TNA) is an artificial genetic polymer. The backbone structure of TNA comprises repeating threose sugars linked by phosphodiester bonds. TNA polymers are resistant to nuclease degradation. TNA can self-assemble by base-pair hydrogen bonding into duplex structures.

Linkage inversions can be introduced into polynucleotides through use of “reversed phosphoramidites” (see, e.g., www.ucalgary.ca/dnalab/synthesis/-modifications/linkages). A 3′-3′ linkage at a terminus of a polynucleotide stabilizes the polynucleotide to exonuclease degradation by creating an oligonucleotide having two 5′-OH termini but lacking a 3′-OH terminus. Typically, such polynucleotides have phosphoramidite groups on the 5′-OH position and a dimethoxytrityl (DMT) protecting group on the 3′-OH position. Normally, the DMT protecting group is on the 5′-OH and the phosphoramidite is on the 3′-OH.

Polynucleotide sequences are displayed herein in the conventional 5′ to 3′ orientation unless otherwise indicated.

As used herein, “sequence identity” generally refers to the percent identity of nucleotide bases or amino acids comparing a first polynucleotide or polypeptide to a second polynucleotide or polypeptide using algorithms having various weighting parameters. Sequence identity between two polynucleotides or two polypeptides can be determined using sequence alignment by various methods and computer programs (e.g., BLAST, CS-BLAST, FASTA, HMMER, L-ALIGN, and the like) available through the worldwide web at sites including, but not limited to, GENBANK (www.ncbi.nlm.nih.gov/genbank/) and EMBL-EBI (www.ebi.ac.uk.). Sequence identity between two polynucleotides or two polypeptide sequences is generally calculated using the standard default parameters of the various methods or computer programs. A high degree of sequence identity between two polynucleotides or two polypeptides is typically between about 90% identity and 100% identity over the length of the reference polypeptide, for example, about 90% identity or higher, preferably about 95% identity or higher, more preferably about 98% identity or higher. A moderate degree of sequence identity between two polynucleotides or two polypeptides is typically between about 80% identity to about 85% identity, for example, about 80% identity or higher, preferably about 85% identity over the length of the reference polypeptide. A low degree of sequence identity between two polynucleotides or two polypeptides is typically between about 50% identity and 75% identity, for example, about 50% identity, preferably about 60% identity, more preferably about 75% identity over the length of the reference polypeptide.

For instance, a nucleic acid sequence of the present disclosure may have a particular sequence identity to a sequence complementary to a poly-U region, and/or a particular sequence identity to a sequence complementary to a sequence at the 5′-end of the negative-strand RNA that starts immediately after the poly-U stretch. This sequence identity may be, for example, 25% or more, 50% or more, 75% or more, 80% or more, 85% or more, 90% or more, 91% or more, 92% or more, 93% or more, 94% or more, 95% or more, 96% or more, 97% or more, 98% or more, or 99% or more.

As used herein, “hybridization,” “hybridize,” or “hybridizing” is the process of combining two complementary single-stranded DNA or RNA molecules so as to form a single double-stranded molecule (DNA/DNA, DNA/RNA, RNA/RNA) through hydrogen base pairing. Hybridization stringency is typically determined by the hybridization temperature and the salt concentration of the hybridization buffer; e.g., high temperature and low salt provide high stringency hybridization conditions. Examples of salt concentration ranges and temperature ranges for different hybridization conditions are as follows: high stringency, approximately 0.01M to approximately 0.05M salt, hybridization temperature 5° C. to 10° C. below Tₘ; moderate stringency, approximately 0.16M to approximately 0.33M salt, hybridization temperature 20° C. to 29° C. below Tₘ; and low stringency, approximately 0.33M to approximately 0.82M salt, hybridization temperature 40° C. to 48° C. below Tₘ. Tₘ of duplex nucleic acid sequences is calculated by standard methods well-known in the art. See, e.g., Maniatis et al. (Molecular Cloning: A Laboratory Manual, 1982, Cold Spring Harbor Laboratory Press: New York); Casey et al. (Nucleic Acids Research, 1977, 4:1539-1552); Bodkin et al. (Journal of Virological Methods, 1985, 10(1):45-52); and Wallace et al. (Nucleic Acids Research, 1981, 9(4):879-894). Algorithm prediction tools to estimate Tₘ are also widely available. High stringency conditions for hybridization typically refer to conditions under which a polynucleotide complementary to a target sequence predominantly hybridizes with the target sequence, and substantially does not hybridize to non-target sequences. Typically, hybridization conditions are of moderate stringency, preferably high stringency.

As used herein, “complementarity” refers to the ability of a nucleic acid sequence to form hydrogen bonds with another nucleic acid sequence (e.g., through canonical Watson-Crick base pairing). A percent complementarity indicates the percentage of residues in a nucleic acid sequence that can form hydrogen bonds with a second nucleic acid sequence. If two nucleic acid sequences have 100% complementarity, the two sequences are perfectly complementary, i.e., all of the contiguous residues of a first polynucleotide hydrogen bond with the same number of contiguous residues in a second polynucleotide.

As used herein, “binding” refers to a non-covalent interaction between macromolecules (e.g., between a protein and a polynucleotide, between a polynucleotide and a polynucleotide, or between a protein and a protein, and the like). Such non-covalent interaction is also referred to as “associating” or “interacting” (e.g., if a first macromolecule interacts with a second macromolecule, the first macromolecule binds to second macromolecule in a non-covalent manner). Some portions of a binding interaction may be sequence-specific (the terms “sequence-specific binding,” “sequence-specifically bind,” “site-specific binding,” and “site specifically binds” are used interchangeably herein). Binding interactions can be characterized by a dissociation constant (Kd). “Binding affinity” refers to the strength of the binding interaction. An increased binding affinity is correlated with a lower Kd.

As used herein, the terms “regulatory sequences,” “regulatory elements,” and “control elements” are interchangeable and refer to polynucleotide sequences that are upstream (5′ non-coding sequences), within, or downstream (3′ non-translated sequences) of a polynucleotide target to be expressed. Regulatory sequences influence, for example, the timing of transcription, amount or level of transcription, RNA processing or stability, and/or translation of the related structural nucleotide sequence. Regulatory sequences may include activator binding sequences, enhancers, introns, polyadenylation recognition sequences, promoters, transcription start sites, repressor binding sequences, stem-loop structures, translational initiation sequences, internal ribosome entry sites (IRES), translation leader sequences, transcription termination sequences (e.g., polyadenylation signals and poly-U sequences), translation termination sequences, primer binding sites, and the like.

Regulatory elements include those that direct constitutive, inducible, and repressible expression of a nucleotide sequence in many types of host cells and those that direct expression of the nucleotide sequence only in certain host cells (e.g., tissue-specific regulatory sequences). In some embodiments, a vector comprises one or more pol III promoters, one or more pol II promoters, one or more pol I promoters, or combinations thereof. Examples of pol III promoters include, but are not limited to, U6 and H1 promoters. Examples of pol 11 promoters include, but are not limited to, the retroviral Rous sarcoma virus (RSV) LTR promoter (optionally with the RSV enhancer), the cytomegalovirus (CMV) promoter (optionally with the CMV enhancer; see, e.g., Boshart et al. (Cell, 1985, 41:521-530)), the SV40 promoter, the dihydrofolate reductase promoter, the β-actin promoter, the phosphoglycerol kinase (PGK) promoter, and the EF1α promoter. It will be appreciated by those skilled in the art that the design of an expression vector may depend on such factors as the choice of the host cell to be transformed, the level of expression desired, and the like. A vector can be introduced into host cells to thereby produce transcripts, proteins, or peptides, including fusion proteins or peptides, encoded by nucleic acid sequences as described herein.

“Gene” as used herein refers to a polynucleotide sequence comprising exons and related regulatory sequences. A gene may further comprise introns and/or untranslated regions (UTRs).

As used herein, the term “operably linked” refers to polynucleotide sequences or amino acid sequences placed into a functional relationship with one another. For example, regulatory sequences (e.g., a promoter or enhancer) are “operably linked” to a polynucleotide encoding a gene product if the regulatory sequences regulate or contribute to the modulation of the transcription of the polynucleotide. Operably linked regulatory elements are typically contiguous with the coding sequence. However, enhancers can function if separated from a promoter by up to several kilobases or more. Accordingly, some regulatory elements may be operably linked to a polynucleotide sequence but not contiguous with the polynucleotide sequence. Similarly, translational regulatory elements contribute to the modulation of protein expression from a polynucleotide.

As used herein, “expression” refers to transcription of a polynucleotide from a DNA template, resulting in, for example, a messenger RNA (mRNA) or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene products.” Expression may include splicing the mRNA in a eukaryotic cell, if the polynucleotide is derived from genomic DNA.

A “coding sequence” or a sequence that “encodes” a selected polypeptide, is a nucleic acid molecule that is transcribed (in the case of DNA) and translated (in the case of mRNA) into a polypeptide in vitro or in vivo when placed under the control of appropriate regulatory sequences. The boundaries of the coding sequence are determined by a start codon at the 5′ terminus and a translation stop codon at the 3′ terminus. A transcription termination sequence may be located 3′ to the coding sequence.

As used herein, the term “modulate” refers to a change in the quantity, degree or amount of a function. Thus, “modulation” of gene expression includes both gene activation and gene repression. Modulation can be assayed by determining any characteristic directly or indirectly affected by the expression of the target gene. Such characteristics include, for example, changes in RNA or protein levels, protein activity, product levels, expression of the gene, or activity level of reporter genes.

“Vector” and “plasmid” as used herein refer to a polynucleotide vehicle to introduce genetic material into a cell. Vectors can be linear or circular. Vectors can contain a replication sequence capable of effecting replication of the vector in a suitable host cell (e.g., an origin of replication). Upon transformation of a suitable host, the vector can replicate and function independently of the host genome or integrate into the host genome. Vector design depends, among other things, on the intended use and host cell for the vector, and the design of a vector of the invention for a particular use and host cell is within the level of skill in the art. The four major types of vectors are plasmids, viral vectors, cosmids, and artificial chromosomes. Typically, vectors comprise an origin of replication, a multicloning site, and/or a selectable marker. An expression vector typically comprises an expression cassette. By “recombinant virus” is meant a virus that has been genetically altered, e.g., by the addition or insertion of a heterologous nucleic acid construct into a viral genome or portion thereof.

As used herein, “expression cassette” and “expression vector” refer to a polynucleotide construct generated using recombinant methods or by synthetic means and comprising regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell. An expression cassette can, for example, be integrated in the genome of a host cell or be present in a vector to form an expression vector.

As used herein, the term “between” is inclusive of end values in a given range (e.g., between about 1 and about 50 nucleotides in length includes 1 nucleotide and 50 nucleotides.

As used herein, the term “amino acid” refers to natural and synthetic (unnatural) amino acids, including amino acid analogs, modified amino acids, peptidomimetics, glycine, and D or L optical isomers.

As used herein, the terms “peptide,” “polypeptide,” and “protein” are interchangeable and refer to polymers of amino acids. A polypeptide may be of any length. It may be branched or linear, it may be interrupted by non-amino acids, and it may comprise modified amino acids. The terms also refer to an amino acid polymer that has been modified through, for example, acetylation, disulfide bond formation, glycosylation, lipidation, phosphorylation, pegylation, biotinylation, cross-linking, and/or conjugation (e.g., with a labeling component or ligand). Polypeptide sequences are displayed herein in the conventional N-terminal to C-terminal orientation, unless otherwise indicated. Polypeptides and polynucleotides can be made using routine techniques in the field of molecular biology. Furthermore, essentially any polypeptide or polynucleotide is available from commercial sources.

The terms “fusion protein” and “chimeric protein” as used herein refer to a single protein created by joining two or more proteins, protein domains, or protein fragments that do not naturally occur together in a single protein.

A “moiety” as used herein refers to a portion of a molecule. A moiety can be a functional group or describe a portion of a molecule with multiple functional groups (e.g., that share common structural aspects). The terms “moiety” and “functional group” are typically used interchangeably; however, a “functional group” can more specifically refer to a portion of a molecule that comprises some common chemical behavior. “Moiety” is often used as a structural description. In some embodiments, a 5′ terminus, a 3′ terminus, or a 5′ terminus and a 3′ terminus can comprise one or more moieties.

The terms “modified protein,” “mutated protein,” “protein variant,” and “engineering protein” as used herein typically refers to a protein that has been modified such that it comprises a non-native sequence (i.e., the modified protein has a unique sequence compared to an unmodified protein).

The terms “subject,” “individual,” or “patient” are used interchangeably herein and refer to any member of the phylum Chordata, including, without limitation, humans and other primates, including non-human primates, such as rhesus macaques, chimpanzees, and other monkey and ape species; farm animals, such as cattle, sheep, pigs, goats, and horses; domestic mammals, such as dogs and cats; laboratory animals, including rabbits, mice, rats, and guinea pigs; birds, including domestic, wild, and game birds, such as chickens, turkeys, and other gallinaceous birds, ducks, and geese; and the like. The term does not denote a particular age or gender. Thus, the term includes adult, young, and newborn individuals as well as males and females.

The terms “effective amount” or “therapeutically effective amount” of a composition or agent, refer to a sufficient amount of the composition or agent to provide the desired response. Preferably, the effective amount will prevent, avoid, or eliminate one or more harmful side-effects. The exact treatment amount required will vary from subject to subject, depending on the species, age, and general condition of the subject, the severity of the condition being treated, and the particular treatment used, mode of administration, and the like. An appropriate “effective” amount in any individual case may be determined by one of ordinary skill in the art using routine experimentation.

“Treatment” or “treating” a particular disease, such as COVID-19, includes: (1) preventing the disease, for example, preventing the development of the disease or causing the disease to occur with less intensity in a subject that may be predisposed to the disease, but does not yet experience or display symptoms of the disease; (2) inhibiting the disease, for example, reducing the rate of development, arresting the development or reversing the disease state; and/or (3) relieving symptoms of the disease, for example, decreasing the number of symptoms experienced by the subject.

“Transformation” as used herein refers to the insertion of an exogenous polynucleotide into a host cell, irrespective of the method used for insertion. For example, transformation can be by direct uptake, transfection, infection, and the like. Nucleic acids and polynucleotides can be introduced into the cell using, for example, viral vectors, nucleofection, gene gun, sonoporation, cell squeezing, lipofection, or chemicals (e.g., cell penetrating peptides).

“Coronavirus” as used herein refers to members of the family Coronaviridae, and therefore includes, but is not limited to, viruses such as Severe Acute Respiratory Syndrome Coronavirus (SARS-CoV), Middle East Respiratory Syndrome Coronavirus (MERS-CoV), and Severe Acute Respiratory Syndrome Coronavirus 2 (SARS-CoV-2; the causative agent of COVID-19).

Agents Targeting 5′ poly-U Sequences

As described herein, the 5′ poly-U tract of the negative-strand intermediates of coronaviruses, such as SARS-CoV-2, can be targeted and blocked using, for instance, a complementary nucleic acid sequence (as blocking the poly-U tract prevents genomic and sub-genomic mRNA synthesis).

In some embodiments, the complementary nucleic acid sequence is a DNA, RNA, or DNA/RNA, and may or may not include one or more abasic sites, base analogs, and/or modified bases, for example. In certain aspects and embodiments, nucleic acid molecules may include one or more modifications (or chemical modifications). Such modifications can be in the nucleotide sugar, nucleotide base, nucleotide phosphate group and/or the phosphate backbone of a polynucleotide.

In certain embodiments, modifications as disclosed herein may be used to increase the in vivo stability of the nucleic acid molecule, particularly the stability in serum, and/or to increase bioavailability of the molecules. Non-limiting examples of modifications include, without limitation, internucleotide or internucleoside linkages; deoxynucleotides or dideoxyribonucleotides at any position and strand of the nucleic acid molecule; nucleic acid (e.g., ribonucleic acid) with a modification at the 2′-position preferably selected from an amino, fluoro, methoxy, alkoxy and alkyl; 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, biotin group, and terminal glyceryl and/or inverted deoxy abasic residue incorporation, sterically hindered molecules, such as fluorescent molecules and the like. Other modified nucleotides may include, for example, 3′-deoxyadenosine, 3′-azido-3′-deoxythymidine, 2′,3′-dideoxyinosine, 2′,3′-dideoxy-3′-thiacytidine, 2′,3′-didehydro-2′,3′-dideoxythymidi-ne and the monophosphate nucleotides of 3′-azido-3′-deoxythymidine, 2′,3′-dideoxy-3′-thiacytidine and 2′,3′-didehydro-2′,3′-dide-oxythymidine.

Also contemplated herein are locked nucleic acid (LNA) nucleotides (e.g., 2′-O, 4′-C-methylene-(D-ribofuranosyl) nucleotides); 2′-methoxyethoxy (MOE) nucleotides; 2′-methyl-thio-ethyl, 2′-deoxy-2′-fluoro nucleotides, 2′-deoxy-2′-chloro nucleotides, 2′-azido nucleotides, and 2′-O-methyl nucleotides. Chemical modifications also include unlocked nucleic acids, or UNAs, which are non-nucleotide, acyclic analogues, in which the C2′-C3′ bond is not present.

Chemical modifications also include terminal modifications on the 5′ and/or 3′ part of the nucleic acids, and are also known as capping moieties. Such terminal modifications are selected from a nucleotide, a modified nucleotide, a lipid, a peptide, and a sugar. Chemical modifications also include “six membered ring nucleotide analogs.” Examples of six-membered ring nucleotide analogs include hexitol and altritol nucleotide monomers.

Chemical modifications also include “mirror” nucleotides which have a reversed chirality as compared to normal naturally occurring nucleotide. Mirror nucleotides may further include at least one sugar or base modification and/or a backbone modification. Mirror nucleotides include, for example, L-DNA (L-deoxyriboadenosine-3′-phosphate (mirror dA); L-deoxyribocytidine-3′-phosphate (mirror dC); L-deoxyriboguanosine-3′-phosphate (mirror dG); L-deoxyribothymidine-3′-phosphate (mirror image dT)) and L-RNA (L-riboudenosine-3′-phosphate (mirror rA); L-ribocytidine-3′-phosphate (mirror rC); L-riboguanosine-3′-phosphate (mirror rG); and L-ribouracil-3′-phosphate (mirror dU).

In some embodiments, modified ribonucleotides include modified deoxyribonucleotides, for example 5′OMe DNA (5-methyl-deoxyriboguanosine-3′-phosphate); PACE (deoxyriboadenosine 3′ phosphonoacetate, deoxyribocytidine 3′ phosphonoacetate, deoxyriboguanosine 3′ phosphonoacetate, deoxyribothymidine 3′ phosphonoacetate).

Nucleobases of the nucleic acids disclosed herein may include unmodified deoxyribonucleotides and ribonucleotides (purines and pyrimidines) such as adenine, guanine, cytosine, thymidine, and uracil. Nucleobases can be modified with natural and synthetic nucleobases, such as thymine, xanthine, hypoxanthine, inosine, 2-aminoadenine, 6-methyl and other alkyl derivatives of adenine and guanine, any “universal base” nucleotides; 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 8-halo, amino, thiol, thioalkyl, hydroxyl and other 8-substituted adenines and guanines, 5-trifluoromethyl and other 5-substituted uncials and cytosines, 7-methylguanine, deazapurines, heterocyclic substituted analogs of purines and pyrimidines, e.g., aminoethoxy phenoxazine, derivatives of purines and pyrimidines (e.g., 1-alkyl-, 1-alkenyl-, heteroaromatic- and 1-alkynyl derivatives) and tautomers thereof, 8-oxo-N6-methyladenine, 7-diazaxanthine, 5-methylcytosine, 5-methyluracil, 5-(1-propynyl)uracil, 5-(1-propynyl) cytosine and 4,4-ethanocytosine). Other examples of suitable bases include non-purinyl and non-pyrimidinyl bases such as 2-aminopyridine and triazines.

Sugar moieties in nucleic acids disclosed herein may include 2′-hydroxyl-pentofuranosyl sugar moiety without any modification. Alternatively, sugar moieties can be modified such as, 2′-deoxy-pentofuranosyl sugar moiety, D-ribose, hexose, modification at the 2′ position of the pentofuranosyl sugar moiety such as 2′-O-alkyl (including 2′-O-methyl and 2′-O-ethyl). i.e., 2′-alkoxy, 2′-amino, 2′-O-allyl, 2′-S-alkyl, 2′-halogen (including 2′-fluoro, chloro, and bromo), 2′-methoxyethoxy, 2′-O-methoxyethyl, 2′-O-2-methoxyethyl, 2′-propargyl, 2′-propyl, ethynyl, ethenyl, propenyl, CF, cyano, imidazole, carboxylate, thioate.

In some embodiments, the pentafuranosyl ring may be replaced with acyclic derivatives lacking the C2′-C3′-bond of the pentafuranosyl ring. For example, acyclonucleotides may substitute a 2-hydroxyethoxymethyl group for-the 2′-deoxyribofuranosyl sugar normally present in dNMPs.

The nucleoside subunits of the nucleic acid disclosed herein may be linked to each other by phosphodiester bond. The phosphodiester bond may be optionally substituted with other linkages. For example, phosphorothioate, thiophosphate-D-ribose entities, triester, thioate, 2′-5′ bridged backbone, PACE, 3′- (or -5′)deoxy-3′- (or -5′)thio-phosphorothioate, phosphorodithioate, phosphoroselenates, 3′- (or -5′)deoxy phosphinates, borano phosphates, 3′- (or -5′)deoxy-3′- (or 5′-)amino phosphoramidates, hydrogen phosphonates, phosphonates, borano phosphate esters, phosphoramidates, alkyl or aryl phosphonates and phosphotriester modifications such as alkylphosphotriesters, phosphotriester phosphorus linkages, 5′-ethoxyphosphodiester, P-alkyloxyphosphotnester, methylphosphonate, and nonphosphorus containing linkages for example, carbonate, carbamate, silyl, sulfur, sulfonate, sulfonamide, formacetal, thioformacetyl, oxime, methyleneimino, methylenemethylimino, methylenehydrazo, methylenedimethylhydrazo and methyleneoxymethylimino linkages.

Nucleic acid molecules disclosed herein may include a peptide nucleic acid (PNA) backbone. The PNA backbone is includes repeating N-(2-aminoethyl)-glycine units linked by peptide bonds. The various bases such as purine, pyrimidine, natural and synthetic bases are linked to the backbone by methylene carbonyl bonds.

Modifications can be made at terminal phosphate groups. Non-limiting examples of different stabilization chemistries can be used, e.g., to stabilize the 3′-end of nucleic acid sequences, including (1) [3-3′]-inverted deoxyribose; (2) deoxyribonucleotide; (3) [5′-3′]-3′-deoxyribonucleotide; (4) [5′-3′]-ribonucleotide; (5) [5′-3′]-3′-O-methyl ribonucleotide; (6) 3′-glyceryl; (7) [3′-5′]-3′-deoxyribonucleotide; (8) [3′-3′]-deoxyribonucleotide; (9) [5′-2′]-deoxyribonucleotide; and (10) [5-3′]-dideoxyribonucleotides. In addition to unmodified backbone chemistries can be combined with one or more different backbone modifications described herein.

Modified nucleotides and nucleic acid molecules as provided herein may include conjugates, for example, a conjugate covalently attached to the nucleic acid molecule. The conjugate may be covalently attached to a nucleic acid molecule via a linker. In one embodiment, a conjugate molecule may include a molecule that facilitates delivery of a nucleic acid molecule into a cell, such as, for example, into a particular type of cell, or into a particular intracellular compartment or vesicle within a cell.

In some embodiments, the nucleic acid contains DNA, and contains a poly-A or oligo(A) region that hybridizes to the 5′ poly-U sequences.

The poly-A or oligo(A) region may be about 100 or less, about 90 or less, about 80 or less, about 70 or less, about 60 or less, about 50 or less, about 40 or less, about 30 or less, about 25 or less, about 20 or less, about 15 or less, about 14 or less, about 13 or less, about 12 or less, about 11 or less, about 10 or less, about 9 or less, about 8 or less, about 7 or less, about 6 or less, about 5 or less, about 4 or less, about 3 or less, or about 2 or less, bases in length.

To further enhance targeting and specificity, and to further reduce off-target effects, the 5′ poly-U sequences may be targeted using a polynucleotide that comprises a poly-A sequence and which further comprises a 5′-end linker region that comprises a sequence complementary to a sequence at the 5′-end of the negative-strand RNA that starts immediately after the poly-U stretch. In this configuration, both the poly-A sequence and the 5′-end linker region sequence will hybridize to coronavirus negative-strand intermediates, such as SARS-CoV-2 negative-strand intermediates.

In other embodiments, the 5′ poly-U stretch may be blocked using an antibody against poly-U. The antibody may be a fragment and/or a derivative of an antibody. Examples of antibody fragments include F(ab′)2, Fab, and Fv. Examples of antibody derivatives include: antibodies to which an amino acid mutation has been introduced in its constant region; antibodies in which the domain arrangement of the constant regions has been modified; antibodies having two or more Fc's per molecule; antibodies consisting only of a heavy chain or only of a light chain; antibodies with modified glycosylation; bispecific antibodies; conjugates of antibodies or antibody fragments with compounds or proteins other than antibodies; antibody enzymes; nanobodies; tandem scFv's; bispecific tandem scFv's; diabodies; and VHHs. The term “antibody” as used herein encompasses such fragments and/or derivatives of antibodies, unless otherwise specified.

The term “monoclonal antibody” conventionally means antibody molecules obtained from a clone derived from a single antibody-producing cell, i.e., a single variety of antibody molecules having a combination of VH and VL with specific amino acid sequences. A monoclonal antibody can also be produced via genetic engineering procedure, by preparing a nucleic acid molecule having a gene sequence encoding the amino acid sequence of the monoclonal antibody protein. A person skilled in the art would also be familiar with techniques for modifying a monoclonal antibody using genetic information about, e.g., H chains, L chains, variable regions thereof, and CDR sequences thereof to thereby improve the binding ability and specificity of the antibody, and techniques for preparing an antibody suitable for a therapeutic agent by altering an animal antibody such as a mouse antibody into a human-type antibody. A human-type monoclonal antibody can also be prepared by sensitizing a non-human transgenic animal carrying a human antibody gene to an antigen, such as poly-U.

In other embodiments, low molecular weight compounds or proteins that target the 5′ poly-U stretch in the negative-strand intermediates may be used. Such low molecular weight compounds or proteins may be identified using, for example, library screening, or in silico modeling.

In other embodiments, aptamers that target the 5′ poly-U stretch in the negative-strand intermediates may be used. Such aptamers may be identified using, for example, library screening, or a Systematic Evolution of Ligands by EXponential enrichment (SELEX) method.

### Delivery of Therapeutic Products and Compositions

Delivery of agents that target the 5′ poly-U sequence may be achieved by a number of methods known to one of ordinary skill in the art. In some embodiments, these agents can be directly introduced into cells. Non-limiting methods to introduce these components into a cell include microinjection, electroporation, nucleofection, lipofection, particle gun technology, and microprojectile bombardment.

Agents that target the 5′ poly-U region, such as nucleic acid molecules, may be delivered to cells using a delivery vehicle, such as a lipid vesicle or carrier. Agents, including nucleic acid molecules, may be delivered or administered to a subject by direct application of the agent (such as a nucleic acid molecule) with a carrier or diluent or any other delivery vehicle that acts to assist, promote or facilitate entry into a cell, including viral sequences, viral particular, liposome formulations, lipofectin or precipitating agents and the like.

Agents that target the 5′ poly-U sequence, such as nucleic acid molecules, can also be administered to cells by a variety of methods known to those of skill in the art, including, but not limited to, encapsulation in liposomes, by iontophoresis, or by incorporation into other vehicles, such as biodegradable polymers, hydrogels, cyclodextrins, poly(lactic-co-glycolic)acid (PLGA) and PLCA microspheres, biodegradable nanocapsules, and bioadhesive microspheres, or by proteinaceous vectors.

In some embodiments, agents that target the 5′ poly-U sequence, such as nucleic acid molecules, can be administered together with polyethylenimine (PEI) to facilitate delivery. In some embodiments, the PEI is a branched PEI. In other embodiments, agents that target the 5′ poly-U sequence, such as nucleic acid molecules, can be administered together with a linear poly methacrylate cationic polymer, such as block copolymer of poly(oligoethylene oxide methacrylate), or block copolymer of poly(dimethylaminoethyl methacrylate).

Agents that target the 5′ poly-U sequence, such as nucleic acid molecules, can be complexed with cationic lipids, packaged within liposomes, or otherwise delivered to target cells or tissues. The nucleic acid or nucleic acid complexes can be locally administered to relevant tissues ex vivo, or in vivo through direct dermal application, transdermal application, or injection, with or without their incorporation in biopolymers. Delivery systems include surface-modified liposomes containing poly(ethylene glycol) lipids (PEG-modified, or long-circulating liposomes or stealth liposomes). These formulations offer a method for increasing the accumulation of drugs in target tissues.

Agents that target the 5′ poly-U sequence, such as nucleic acid molecules, can be formulated or complexed with polyethylenimine (e.g., linear or branched PEI) and/or polyethylenimine derivatives, including for example polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives, grafted PEIs such as galactose PEI, cholesterol PEI, antibody derivatized PEI, and polyethylene glycol PEI (PEG-PEI), for example.

Agents that target the 5′ poly-U sequence, such as nucleic acid molecules, can be complexed with membrane disruptive agents. The membrane disruptive agent or agents and the nucleic acid molecule may also be complexed with a cationic lipid or helper lipid molecule.

Delivery systems may include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone).

Compositions, methods and kits disclosed herein may include an expression vector that includes a nucleic acid sequence encoding at least one nucleic acid molecule of the invention in a manner that allows expression of the nucleic acid molecule. Methods of introducing nucleic acid molecules or one or more vectors capable of expressing the nucleic acid molecule in the environment of the cell will depend on the type of cell and the make up of its environment.

Nucleic acid molecules that hybridize to 5′ poly-U-containing RNAs of coronavirus (e.g., from SARS-CoV-2) may be expressed from transcription units inserted into DNA or RNA vectors. Recombinant vectors can be DNA plasmids or viral vectors. Nucleic acid molecule-expressing viral vectors can be constructed based on, but not limited to, adeno-associated virus, retrovirus, adenovirus, or alphavirus. The recombinant vectors capable of expressing the nucleic acid molecules can be delivered as described herein, and persist in target cells. Alternatively, viral vectors can be used that provide for transient expression of nucleic acid molecules. Such vectors can be repeatedly administered as necessary. Delivery of nucleic acid molecule-expressing vectors can be systemic, such as by intravenous or intramuscular administration.

An expression vector may include one or more of the following: a) a transcription initiation region (e.g., eukaryotic pol I, II or III initiation region); b) a transcription termination region (e.g., eukaryotic pol I, II or III termination region); c) an intron and d) a nucleic acid sequence encoding at least one of the nucleic acid molecules, wherein said sequence is operably linked to the initiation region and the termination region in a manner that allows expression and/or delivery of the nucleic acid molecule.

Transcription of the nucleic acid molecule sequences can be driven from a promoter for eukaryotic RNA polymerase I (pol I). RNA polymerase II (pol II), or RNA polymerase III (pol III).

Polymeric nanocapsules or microcapsules facilitate transport and release of the encapsulated or bound agents, such a nucleic acids, into a cell. They include polymeric and monomeric materials, such as polybutylcyanoacrylate. Nucleic acid molecules may also be formulated as a microemulsion. A microemulsion is a system of water, oil and amphiphile which is a single optically isotropic and thermodynamically stable liquid solution. Delivery formulations can also include water soluble degradable crosslinked polymers.

The useful dosage to be administered and the particular mode of administration will vary depending upon such factors as the cell type, or for in vivo use, the age, weight and the particular animal and region thereof to be treated, the particular agent (e.g., a nucleic acid) and delivery method used, the therapeutic or diagnostic use contemplated, and the form of the formulation, for example, suspension, emulsion, micelle or liposome, as will be readily apparent to those skilled in the art.

When lipids are used to deliver the nucleic acid, the amount of lipid compound that is administered can vary and generally depends upon the amount of nucleic acid being administered. For example, the weight ratio of lipid compound to nucleic acid is preferably from about 1:1 to about 30:1, with a weight ratio of about 5:1 to about 10:1 being more preferred.

A suitable dosage unit of nucleic acid molecules may be in the range of 0.001 to 0.25 milligrams per kilogram body weight of the recipient per day, or in the range of 0.01 to 20 micrograms per kilogram body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day.

The administered agents that target the 5′ poly-U-containing RNAs (e.g., from SARS-CoV-2) can be used to treat individuals infected with, or suspected of being infected with, a coronavirus, such as SARS-CoV-2.

The administration of the agents and compositions of the present disclosure to subjects may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally. The agents or compositions can be administrated in one or more doses. In some embodiments, an effective amount is administrated as a single dose. In other embodiments, an effective amount is administrated as more than one dose, over a period time. The determination of optimal ranges of effective amounts of a given cell type for a particular disease or condition is within the skill of those in the art.

Treatments of the present disclosure can be provided in conjunction with, before, or after, vaccination, and/or supportive care, such as supplemental oxygen, mechanical ventilation, and steroids.

### Detection Methods

By targeting a target unique to coronaviruses, such as SARS-CoV-2, the 5′ poly-U stretch in the negative-strand intermediates can be used as a marker for a coronavirus infection, such as SARS-CoV-2 infection; and/or as a marker for the presence of a coronavirus, such as SARS-CoV-2, in a sample. Therefore, nucleic acids and compositions of the present disclosure also have utility in the field of coronavirus detection, diagnosis, and management, such as SARS-CoV-2 detection, diagnosis, and management.

Agents as described above for use in treatment can also be used in such detection methods, as long as their binding to the 5′ poly-U stretch leads to a detectable change. For instance, poly-A-containing nucleic acid molecules that specifically hybridize to the 5′ poly-U stretch can be labeled, and their hybridization to the 5′ poly-U stretch can be determined by a change in a detectable signal. For instance, poly-A-containing nucleic acid molecules can be labeled with a fluorescent dye or label.

Fluorescence broadly refers to the process or the result of the emission of light by a substance that has absorbed light or other electromagnetic radiation. Fluorophores or fluorescent dyes are chemical compounds or moieties that can re-emit light upon light excitation. Fluorophores typically contain several combined aromatic groups, or plane or cyclic molecules with several π bonds. A fluorophore absorbs light energy of a specific wavelength and re-emits light at a longer wavelength. When a fluorophore is excited at a particular wavelength, it is promoted to an excited state. In the absence of a quencher, the excited dye emits light in returning to the ground state. When a quencher is present in physical proximity, the excited fluorophore can return to the ground state by transferring its energy to the quencher, without the emission of light.

Different types of quenchers exist. One quenching mechanism relies on the ability of the fluorophore to transfer energy to a second fluorophore by fluorescence resonance energy transfer (FRET). This returns the fluorophore to the ground state and generates the quencher excited state. The quencher then returns to the ground state through emissive decay (fluorescence). In order for this to happen, the emission spectrum of the fluorophore must overlap with the absorption spectrum of the second fluorophore (quencher). One example of such the fluorophore/quencher pair is fluorescein (used as the fluorescent reporter dye) and rhodamine as the quencher (FAM/TAM probes).

However, quencher fluorescence can increase background noise due to overlap between the quencher and reporter fluorescence spectra. Dark quenchers are dyes with no native fluorescence. Dark quenchers return from the excited state to the ground state via non-radiative decay pathways, without the emission of light. In dark decay, energy is given off via molecular vibrations (heat). With the typical μM or less concentration of probe, the heat from radiationless decay is too small to affect the temperature of the solution. Dark quenchers do not occupy an emission bandwidth and allow multiplexing, when two or more reporter-quencher probes are used together. BHQ quenchers are examples of dark quenchers. Thus, a dark quencher, a substance or moiety that absorbs excitation energy from a fluorophore and dissipates the energy as heat, may be used in embodiments of the present disclosure.

Non-limiting examples of detection methods include optical detection, spectroscopic detection, electrostatic detection and electrochemical detection. Accordingly, an agent or nucleic acid molecule described herein may be detected by detecting signals (e.g., signals indicative of an optical property, a spectroscopic property, an electrostatic property or an electrochemical property of the agent, nucleic acid molecule, or an associated detectable label) that are indicative of the presence or absence of the nucleic acid molecule. Optical detection methods include, but are not limited to, visual inspection (e.g., detection via the eye, observing an optical property or optical event without the aid of an optical detector), fluorimetry, chemiluminescence imaging, fluorescence resonance energy transfer (FRET) and UV-vis light absorbance. Spectroscopic detection methods include, but are not limited to, mass spectrometry, nuclear magnetic resonance (NMR) spectroscopy, Raman spectroscopy, and infrared spectroscopy. Electrostatic detection methods include, but are not limited to, gel based techniques, such as, for example, gel electrophoresis (e.g., agarose gel or polyacrylamide gel electrophoresis).

In some embodiments, detection of a nucleic acid molecule described herein may be achieved with the aid of a detectable label, such as a dye, protein, or conjugate. A detectable label may be linked or coupled with a nucleic acid molecule covalently and/or non-covalently (e.g., including intercalation of a double-stranded nucleic acid molecule). Non-limiting examples of detectable labels include optically-responsive species (e.g., optically-responsive dyes, optically-responsive oligonucleotide probes (e.g., TaqMan probes, TaqMan Tamara probes, TaqMan MGB probes, Lion probes, molecular beacons)) and radiolabels.

In some embodiments, one or more of the reagents (e.g., a nucleic acid probe) described herein can comprise a detectable label and/or comprise the ability to generate a detectable signal (e.g., by catalyzing a reaction converting a compound to a detectable product). Detectable labels can comprise, for example, a light-absorbing dye, a fluorescent dye, or a radioactive label. Detectable labels, methods of detecting them, and methods of incorporating them into reagents (e.g., antibodies and nucleic acid probes) are well known in the art.

In some embodiments, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels can be primary labels (where the label comprises a moiety that is directly detectable or that produces a directly detectable moiety); or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to, radioisotopes, bioluminescent compounds, chromophores, antibodies, chemiluminescent compounds, fluorescent compounds, metal chelates, and enzymes.

In some embodiments, a detectable label may be an optically-responsive dye (e.g., a fluorescent dye) that generates (or fails to generate a signal) when subjected to the appropriate conditions. Non-limiting examples of dyes include SYBR green, SYBR blue, DAPI, propidium iodine, Hoeste, SYBR gold, EvaGreen, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), SYBR Green, Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5- (or 6-) iodoacetamidofluorescein, 5-{[2(and 3)-5-(Acetylmercapto)-succinyl]amino}fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores.

In certain embodiments, the 5′ poly-U stretch in the negative-strand intermediates can be detected using a nucleic amplification reaction that amplifies a nucleic acid sequence that contains a sequence corresponding to all or a part of the poly-U stretch (e.g., polymerase chain reaction, RT-PCR).

For instance, in some embodiments, a primer comprising a portion of the SARS-CoV-2 genome upstream of the poly-A sequence (or a primer containing the equivalent cDNA sequence) can be used as a primer to bind to the negative-strand intermediates to initiate first strand synthesis. In some embodiments, this primer sequence lies within the 3′-untranslated region (3′-UTR) of the SARS-CoV-2 genome. Second-strand synthesis can then be performed using, for example, an oligo(dT) primer.

Other known amplification methods which can be utilized herein include, but are not limited to, “NASBA” or “3SR” techniques; Q-beta amplification; strand displacement amplification; target mediated amplification; ligase chain reaction (LCR); self-sustained sequence replication (SSR); and transcription amplification.

Methods for detecting, characterizing, and/or quantitating, nucleic acid sequences are known to persons skilled in the art, and include, but are not limited to, for example, PCR procedures, RT-PCR, quantitative PCR or RT-PCR, Northern blot analysis, differential gene expression, RNA protection assay, microarray analysis, hybridization methods, serial analysis of gene expression (SAGE), hybridization based on digital barcode quantification assays, multiplex RT-PCR, digital drop PCR (ddPCR), qRT-PCR, qPCR, UV spectroscopy, DNA sequencing, RNA sequencing, next-generation sequencing, including RNAseq, lysate-based hybridization assays utilizing branched DNA signal amplification, such as the QuantiGene 2.0 Single Plex, and branched DNA analysis methods.

Non-limiting examples of nucleic acid sequencing techniques, e.g., for DNA sequencing and RNA sequencing, include Maxam-Gilbert sequencing, Sanger sequencing (i.e., chain-termination), sequencing-by-synthesis (SBS), sequencing-by-ligation, pyrosequencing, single-molecule real-time sequencing, MiSeq sequencing, massively parallel signature sequencing (MPSS), polony sequencing, 454 sequencing, nanopore sequencing. The present disclosure also encompasses, but is not limited to, next-generation sequencing technologies.

Non-limiting examples of next-generation sequencing technologies include, for example, Ion Torrent, Illumina, SOLiD, 454; Massively Parallel Signature Sequencing solid-phase, reversible dye-terminator sequencing; and DNA nanoball sequencing. Digital barcode quantification assays can include the BeadArray (Illumina), the xMAP systems (Luminex), the nCounter (Nanostring), the High Throughput Genomics (HTG) molecular, BioMark (Fluidigm), or the Wafergen microarray. Assays can include DASL (Illumina), RNA-Seq (Illumina), TruSeq (Illumina), SureSelect (Agilent), Bioanalyzer (Agilent) and TaqMan (ThermoFisher).

In general, PCR describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e., each primer is specifically designed to be complementary to a strand of the genomic locus to be amplified. The mRNA level of a gene can be determined by reverse-transcription (RT) PCR and by quantitative RT-PCR (QRT-PCR), or real-time PCR methods. Methods of RT-PCR and QRT-PCR are well known in the art. The nucleic acid sequences of exemplary marker genes are set forth herein. Accordingly, a skilled artisan can design an appropriate primer based on the disclosed sequences for determining the mRNA level of the respective marker gene.

Nucleic acid and ribonucleic acid (RNA) molecules can be isolated from a sample using any of a number of procedures, which are well-known in the art, the particular isolation procedure chosen being appropriate for the particular biological sample.

### EXAMPLES

Non-limiting embodiments of the present invention are illustrated in the following Examples. Efforts have been made to ensure accuracy with respect to numbers used (e.g., amounts, concentrations, percent changes, and the like), but some experimental errors and deviations should be accounted for.

Unless indicated otherwise, temperature is in degrees Centigrade and pressure is at or near atmospheric. It should be understood that these Examples are given by way of illustration only and are not intended to limit the scope of what the inventor regards as various embodiments of the present invention. Not all of the following steps set forth in each example are required nor must the order of the steps in each example be as presented.

### Example 1

### Design of DNA Oligonucleotide Complementary to MHV-A59 5′ End of the Antigenome (Minus RNA Genome)

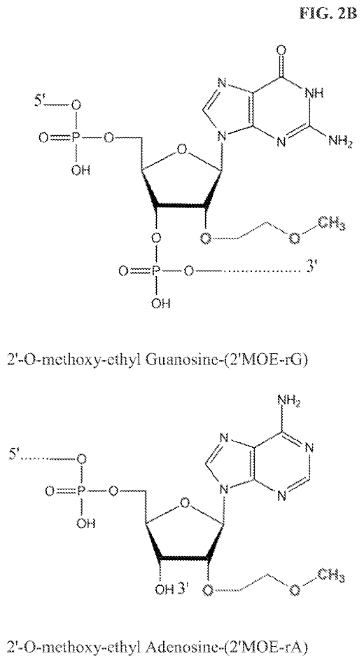

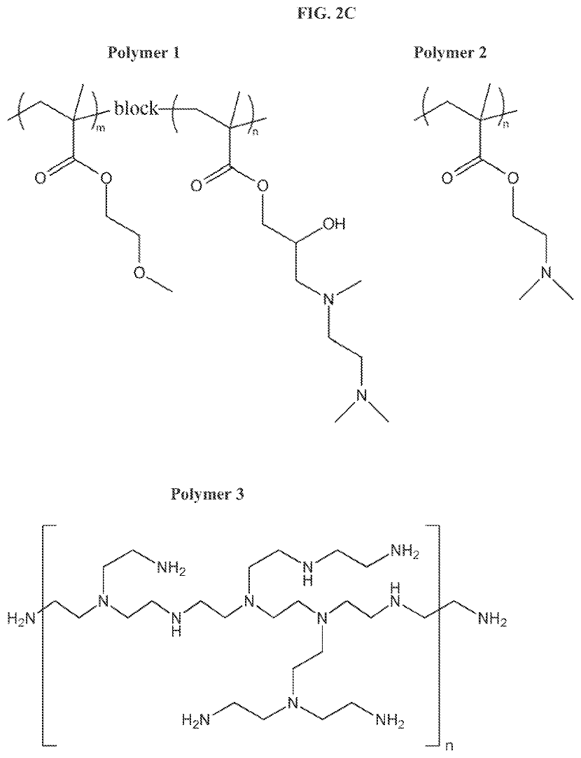

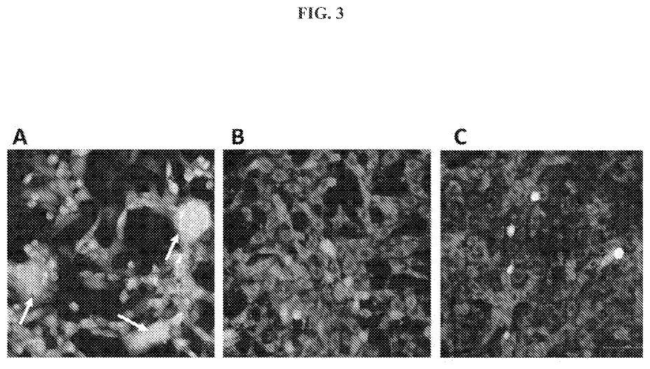

This Example illustrates the design and synthesis of nucleic acid molecules targeting the 5′-poly(U) tract of SARS-CoV-2. Using the whole genome of sequences of MHV-A59 (accession #NC_001846), two oligonucleotides were designed that were complementary to the polyUs at the 5′ end of the minus strand of the virus genome (FIG. 2A). The oligonucleotides were designed with twenty one 3′-polyAs and an additional 11 bases complementary to the flanking polyAs so that the designed oligos would be able to distinguish the 5′-polyUs from the 3′-polyUs. Due to the possible use of the oligonucleotide as a primer to amplify flanking sequence, a frameshift mutation was introduced by deleting a base in the Bison Oligo-2 3′UTR flaking sequence. Additional modifications were done to the oligonucleotides to provide increased resistance to nuclease degradation, reduced toxicity, and to provide increased affinity for binding to complementary RNA (FIG. 2B). Though these kinds of modification have been used in antisense oligonucleotide technology to force usage of alternative polyadenylation site selection and to increase the abundance of message, here the use of a poly-A oligonucleotide, rather than a gene sequence-targeted oligonucleotide, will minimize any such effect on the genome. Though there are 11 flanking bases running into the 3′ UTR next to the N gene sequence of the virus, lack of any cryptic polyadenylation signal in the vicinity of the targeted sequence reduces any possibility of the use of an alternative polyadenylation site. FIG. 2C shows the molecular structures of block copolymer-based oligo carrier nanoparticles which are described in detail below

### Example 2

### Oligo Carrier Nano-Particles

This Example demonstrates the effectiveness of the Polymer1 and Polymer2 system as a delivery vehicle. Oligonucleotides, due to their anionic charge, size and nuclease sensitivity, have minimal cellular penetration, rapid elimination half-life, and can also induce an immunological response. To overcome these challenges of oligonucleotide delivery, cationic polymers were used to complex and transfect the oligonucleotides to the infected cells.

To determine if the delivery vehicle would impact the efficacy of the DNA oligonucleotide, the oligo was formulated with either a lipid-based delivery agent (lipofectamine) or a polymer-based system. The oligonucleotide was complexed via electrostatic interactions of the negatively-charged DNA backbone and cationic charge on the lipid/polymer systems. The lipid-based oligonucleotide delivery systems are currently employed in the mRNA-based delivery of SARS-Cov2 vaccines, albeit with unknown lipid structures. The Polymer1 and Polymer2 system, are is a block copolymer with a first block that provides a hydrophilic biocompatible shell of oligoethylene oxide methacrylate and second block comprised of a hydrophilic tertiary amine methacrylate, polymer2 is a linear poly (dimethylamino methacrylate) and were prepared. The polymer-based delivery systems were found to provide superior transfection (data not shown) and were used in subsequent examples.

### Example 3

### Oligonucleotide-Induced Inhibition of Infectious Virion Development

This Example demonstrates that oligonucleotides interfere with the development of the +RNA genome and the sub-genomic mRNAs. Mouse hepatitis virus (MHV) serves as a model for the family of enveloped plus RNA viruses from Coronaviridae and the MHV-A59 strain is the prototype coronavirus (CoV). Strain MHV-A59, harboring an eGFP fluorescent tag that was inserted by replacing a pseudogene-ORF4, was used. Serial dilution of the stock virus was used to infect the murine fibroblast cell line 17CL-1.

The murine 17CL-1 cell line (derived from 3T3 cells) was obtained through BEI Resources, NIAID, NIH, catalog number: NR-53719. The cells were maintained as monolayer cultures in Minimum Essential Medium (MEM; Sigma Aldrich-M 4655) containing 10% fetal bovine serum (FBS; Life Technologies), 100 IU/ml of penicillin, and 100 μg/ml of streptomycin (Both from Life Technologies) in a 37° C. humified incubator supplemented with 5% CO2. MHV strain A59-eGFP, which express the Enhanced Green Fluorescent Protein (eGFP) inserted in place of the Ns4 gene, was obtained through BEI Resources, NIAID, NIH, catalog number: NR-53716.

For infection, the 70-80% confluent 17CL-1 cells were pretreated with the oligo (6 μg) plus polymer (1 μg oligo/μg of polymer) or polymer only for 1 h at 37 C inside 5% CO2 supplemented incubator. Cells were then infected with the MHV-A59-eGFP at 0.01 MOI. After one hour of adsorption, the media were removed and the cells were washed three times with PBS buffer. New MEM media with oligo plus polymer or polymer only was added to the virus treated cells and was incubated for 24 h. Trypsinized 17CL-1 cells from non-treated wells were pelleted (10\hat{ }6 cells/ml) and then labelled with diluting dye (BioTracker™ NIR680 Cytoplasmic Membrane Dye Live Cell Dye, Sigma-Aldrich, Cat. #SCT112) 1:2000 in culture medium for a final dye concentration of 1 uM. After 20 minutes of incubation inside 37 C cell culture incubator, cells were pelleted at 350×g for 5 minutes. The pelleted cells were washed 3 times with cell culture medium and 0.25×10\hat{ }6/ml resuspended secondary cells along with oligo plus polymer or polymer only added to the primary cells already infected with the virus and the cells were assayed at the indicated times of incubation.

In order to demonstrate that oligonucleotide would interfere with the development of the +RNA genome and the sub-genomic mRNAs, which are synthesized in specialized Replication-Transcription Centers (RTCs), the primary infection-based development of infectious virions, which are released from the infected cells into the media, were assayed to demonstrate that the oligonucleotides interfere with the development of infectious virions. NIR680 labelled secondary cells (magenta cells) were added to the primary cells 24 h post infection and assayed for any virus-induced infectious effects in the secondary cells (FIG. 3). In control cells that were treated with the polymer alone, clear infectiousness of the virus was observed in the secondary magenta colored cells, which were mostly clustered in specific foci (FIG. 3A). Additionally, extensive cell-to-cell fusions to form syncytium-another indicator of infectious virion production were evident (arrow, FIG. 3A). On the other hand, within the oligonucleotide treated cells, only a few of the GFP-positive secondary magenta colored cells were seen and hardly any syncytium formation was seen within the secondary cells (FIG. 3B, 3C). In a few instances, some secondary cells in the presence of oligonucleotide did show GFP positive cells but as no cytopathic effects or any syncytium were evident in those GFP positive cells (data not shown), thus suggesting that the GFP positive cells in the oligonucleotide treated cells might not be producing fully formed functional virions.

### Example 4

Oligonucleotide Treatment Interferes with the Release of Nucleocapsid Protein from the Host Cells

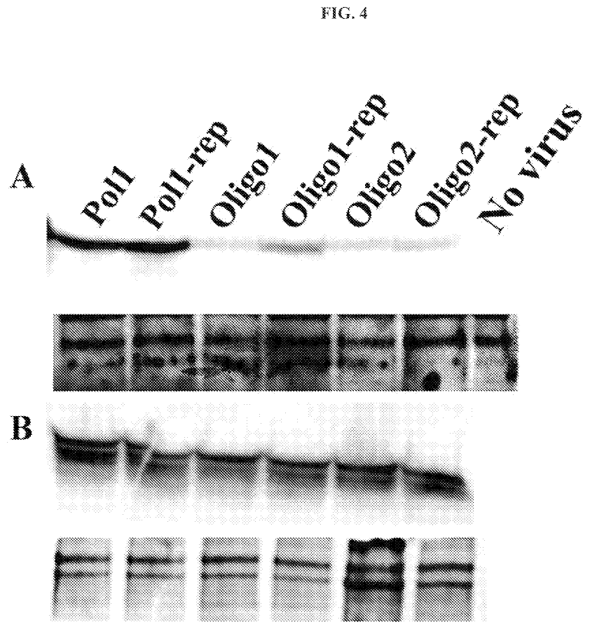

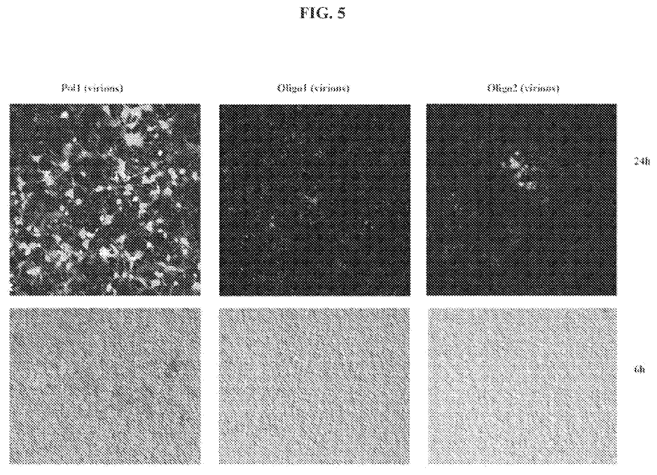

This Example demonstrates that the oligonucleotides are preventing the generation of fully functional virions in cells depicted in FIG. 3. Virion proteins were isolated from the same control and treated cell wells (from around the coverslip periphery) and media from the respective wells. Western assay was used to detect viral nucleocapsid protein N. The N protein is the only viral structural protein that is known to bind to the RTCs and is essential in the incorporation of the viral genome into a fully functional virus particle. FIGS. 4A and 4B show the results obtained from the released virions in the media (FIG. 4A) and from the virus infected cells treated with or without the oligonucleotide (FIG. 4B). While both the biological replicates of released viral proteins in the media treated only with polymer1 showed extensive production of N-proteins, the released virions from cells treated with the oligonucleotides produced a non-significant level of N-protein. No N-protein was detected in media obtained from cells that were not treated with virus. While cells treated with the oligonucleotide show slightly lower levels of N-protein production, compared to that of cells treated with the polymer only (FIG. 4B), the absence of N-proteins in the released virions indicates that the oligonucleotide treatment was indeed able to prevent the formation of the key structural protein required for the formation of infectious virions.

For the Western analysis, cells were washed three times with cold PBS. Lysates were isolated in lysis buffer (Cell Signaling, Danvers, MA) supplemented with protease inhibitor (Sigma-Aldrich), phosphatase inhibitor cocktail A and B (Santa Cruz Biotechnology, Dallas, TX), and N-Ethylamaleimide (Sigma-Aldrich, St. Louis, MO). The concentration of all proteins was measured using Bio-Rad's Bradford dye following the manufacturer's instructions. Lysates from media supernatants were prepared by three quick freeze-thaw cycles of the media. After 50 microgram of cell lysates or equal amount of supernatant lysates with 1× loading dye (Biorad, Hercules, CA) supplemented with 50 μl/ml βME (beta-mercaptoethanol) were boiled for 10 minutes, chilled lysates were loaded on the BioRad's 4-12% precast Bis-Tris polyacrylamide gel, Proteins were then transferred onto a precut PVDF membrane (Biorad) using Biorad electro-transfer set up. After transfer, the gel was incubated overnight with Coomassie dye to stain the left-over proteins as loading control. The membrane was blocked with 5% nonfat milk in PBST (1×PBS with 0.1% Tween 20) for one hour, three washes were done for 10 minutes each by using PBST (1×PBS with 0.1% Tween 20). Washed membrane was incubated overnight at 4 C with the indicated diluted primary antibody, followed by incubation with a horseradish peroxidase coupled secondary antibody. After washing three times with PBST, the protein expression was visualized by Biorad's Clarity ECL reagent using Protein Simple Fluor-Chem M system.

### Example 5

Inhibition of Infectivity by Virions Obtained from Oligo-Treated Cells

This Example confirms that the released virions from the oligonucleotide-treated cells are not infectious by determining the infection potential of the virions obtained from the media was assayed. The same media supernatants used in the western assay in FIG. 4 were serially diluted and used to infect the 17CL-1 cells for 24 h and NIR680-labelled secondary cells were subsequently added. The cell lysates western showed almost similar levels of N-protein production across the control and treatments presumably from the primary infection (FIG. 4B). In order to evaluate whether similar levels of infectious virions were released in the medium, serial dilutions of the medium supernatant were made without normalizing for the virus amount. FIG. 5 shows the infection from the 10⁻³ dilution of medium containing the released virions from the respective treatment conditions. During the infection assay, no oligonucleotide or polymer was added. As can be seen in FIG. 5, the 10⁻³ dilution of the media containing the released virions from the polymer-treated cells were successful in infecting the cells, as evident by the cytopathic effect as early as 6 h post-addition of the secondary cells (FIG. 5 lower panel). The media from the oligonucleotide-treated cells, on the other hand, did not show cytopathic effects during the early infection phase. Virion infectivity at 24 h post-secondary cell addition was also assayed. At 24 h, the media containing virions released from polymer treated cells caused infection (GFP positive) in almost all of the magenta colored secondary cells (FIG. 5, upper panel). On the other hand, only few magenta colored secondary cells got infected when media containing virions released from the oligonucleotide-treated cells was used. the released virions media. Together, this infectivity assay confirms that treatment with the oligo prevented the release of infectious virions into the media.

### Example 6

### Oligonucleotide Inhibits Development of Double-Stranded RNA Intermediaries

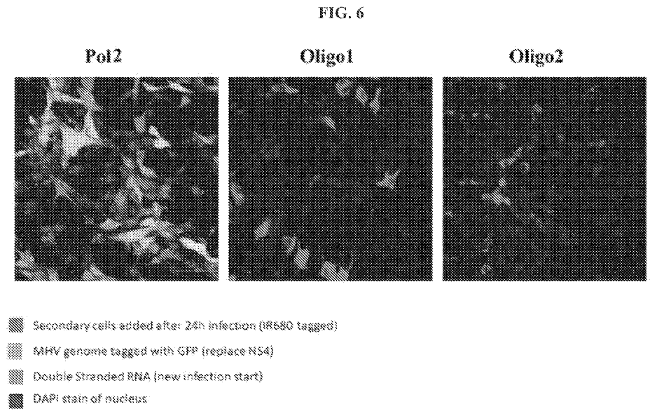

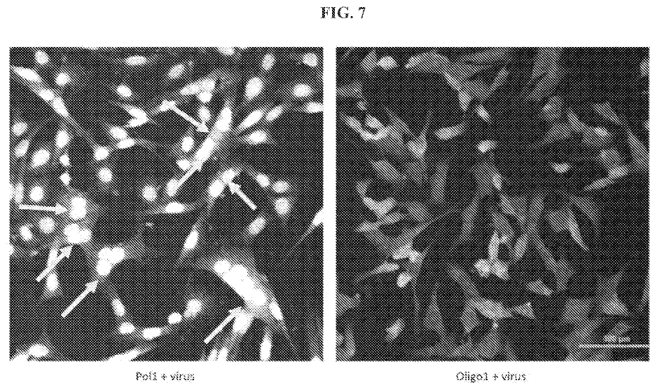

This Example demonstrates that the oligos can prevent the formation of double-stranded RNA intermediaries. RTCs in virus-infected cells were assayed to test for the presence of replicating virions in the oligonucleotide-treated conditions. In order to support viral replication and RNA synthesis, coronaviruses are known to develop double membrane vesicles (DMVs) by modifying cytoplasmic membranes in the perinuclear area to anchor RTCs. During the active infection phase, coronaviruses produce various complete- and partially dsRNA intermediaries. These ds-RNA intermediaries are considered to be a marker for the location of active viral RNA synthesis in infected cells. To demonsrate that MHV-A59 is able to establish active RNA synthesis processes in the presence of the oligonucleotide. As shown in FIG. 6, use of ds-RNA specific antibody indicated that the oligonucleotide significantly inhibited the development of ds-RNA intermediaries, while without the oligonucleotide, the virus actively developed replication/transcription intermediaries, suggesting active infection. Without the presence of active RNA synthesis mechanisms in a plus RNA virus such as MHV-A59, the formation of fully functional virions unlikely to occur. Therefore, it is likely that the oligonucleotide-induced inhibition of ds-RNA intermediary development is the underlying mechanism by which infection-incapable virions are produced in treated conditions.

The antibody directed against double-stranded RNA was obtained from UniQuest Pty Limited—the commercialization company of the University of Queensland, Queensland 4072, Australia, (Cat #MMABA 2G4). Monoclonal Anti-Murine Coronavirus Nucleocapsid (N) Protein, Clone 1.16.1 (Cat #NR-45106) was obtained from BEI Resources, NIAID, NIH.

For the immunofluorescence assay, the cells on glass coverslip in 12 well plate were incubated with the treatments as described in the Infection method section. After the indicated time frame, the cells were washed 3× with PBS, fixed with 4% PFA-PBS for 15 minutes. The permeabilization was done at room temperature with 0.5% Triton X-100 in PBS (pH 7.4) for 10 min. Cells were washed 3× with ice cold PBS. For blocking, the cells were incubated with 5% BSA, 22.52 mg/ml glycine in PBST (PBS with 0.1% Tween 20) for 30 min at room temperature. The washed cells were incubated in a humified 4 C chamber overnight with the anti-dsRNA antibody (1:250) in PBST with 1% BSA. The cells were washed three times in PBS, 5 min each wash and then Incubated cells with the anti-mouse TRITC conjugated secondary antibody (1:1000) in 1% BSA for 1 h at room temperature in the dark. After washing three times with PBST for 5 minutes each, the cells were mounted on slide using Antifade mounting media with DAPI (4-6-diamidino2-phenylindole; ThermoFisher). After sealing the coverslips with clear nail polish, the slides were left at RT overnight for curing. Imaging was performed using an Eclipse Ti 2000 laser-scanning confocal microscope (Nikon CSU series Spinning Disk confocal microscope) with DAPI, FITC, TRITC, and Cy5 filter sets.

### Example 7

Bison Oligo-1 Targeting the MHV 5′ polyU on Minus RNA Strand Prevents MHV Induced Syncytium Formation in Mouse 17CL-1 Cells

This Example demonstrates that Bison Oligo-1 can prevent the formation of syncytium resulting from coronavirus infection. Cells on coverslips were infected with Mouse coronavirus (MHV) at 0.1 MOI for 1 hour. Cells were washed 3× with PBS and treated with the Polymer1 only (left panel) and with polymer1 (6 ug/ml) and Oligo-1 (6 ug/ml) for 24 hours. The cells were washed and then fixed with 4% paraformaldehyde for 10 minutes. Cells were mounted on slide with DAPI mounting media. Cells were observed under a Nikon Confocal Microscope (FIG. 7).

### Example 8

Bison Oligo-1 Targeting the MHV 5′ polyU on Minus RNA Strand Prevented MHV Induced Inhibition of Cell Cycle Marker-CDC25A

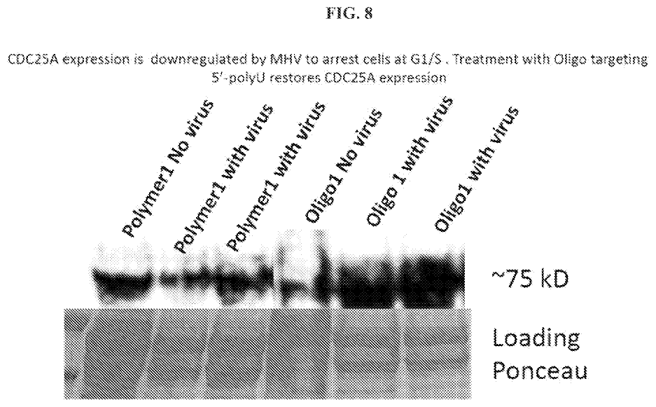

This Example demonstrates that Bison Oligo-1 can prevent the inhibition of cell cycle Marker-CDC25A by MHV. 17CL-1 cells were infected with mouse coronavirus (MHV) at 0.1 MOI for 1 hour. Cells were washed 3× with PBS and then cells were treated as indicated in FIG. 8. After 48 h, cell lysates were separated by SDS-PAGE (50 μg loaded in each lane), transferred to nitrocellulose membranes (Amersham Inc., Piscataway, NJ, USA), and blocked with 5% nonfat milk for 60 min at room temperature. Membranes were incubated overnight at 4° C. with primary antibody (Anti-cell division cycle 25 homolog A (S. pombe) antibody produced in rabbit: Milipore-Sigma Cat #SAB4300430) followed by incubation with a horseradish peroxidase-coupled secondary antibody and detection with enhanced chemiluminescence (Pierce, Rockford, IL, USA). The results are shown in FIG. 8.

### Example 9

### Image-Based Flowcytometry Shows Oligo Induced Rescue of Cell Cycle Arrest by MHV. Arrest of Cell Cycle by MHV

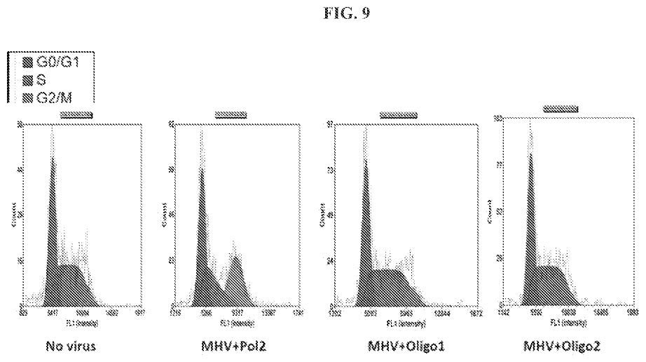

This Example demonstrates that both Bison Oligo-1 and Bison Oligo-2 are capable of rescuing the cell cycle arrest that can result from coronavirus infection. Mouse 17CL-1 cells were infected with mouse coronavirus (MHV) at 0.1 MOI for 1 hour. Cells were washed 3× with PBS and then cells were treated for 24 h as indicated in FIG. 9. Cells were washed with PBS 2× and then cells were fixed with ice-cold 70% ethanol for 30 minutes. Cells were stored in 70% ethanol at 4° C. refrigerator for 72 h. After washing 2× with PBS, cells were treated with ribonuclease (50 ul of a 100 ug/ml stock). Then cells were stained with 100 ul of propodium iodide (PI) from 50 ug/ml stock and were kept at 37° C. for 30 minutes in the dark. The labeled cells were counted in a Nexcelom Vision Cellometer equipped with Brightfield & 2 Fluorescence Channels Filter Set 101: Excitation/Emission Peak: 475 nm/535 nm and Filter Set 202: Excitation/Emission Peak: 525 nm/595 nm). The stained cell samples (20 μl) were pipetted into a Nexcelom counting chamber and inserted into the image cytometer. Bright-field and fluorescent images were then captured and analyzed with De Novo FCS 6 Express software to plot the cell cycles. The software fitted models with the lowest chi-square value were selected to depict in the image. The image represents one of the three replicates done on the samples.

Table 1 shows the percentages of the cells in different stages of cell cycles in samples depicted in FIG. 9. The MHV treated cells without any oligo treatment shows a high percentages of cells in the G2 stage. Surprisingly, Bison Oligo-2 was more effective than Bison Oligo-1 in rescuing cell cycle arrest.

TABLE 1

| | G1 | G1 | | G2 | G2 | | | |
|---|---|---|---|---|---|---|---|---|
| Treatment | Mean | CV | % G1 | Mean | CV | % G2 | % S | G2/G1 |
| No Virus | 5399.4 | 9.5 | 41.8 | 10819.61 | 7.25 | 9.19 | 49.01 | 2.01 |
| No Oligo | | | | | | | | |
| Virus | 5599.3 | 9.45 | 39.88 | 9683.54 | 10.85 | 31.92 | 28.2 | 1.93 |
| No Oligo | | | | | | | | |
| Virus | 4626.86 | 8.53 | 39.72 | 9590.14 | 7.66 | 9.96 | 50.32 | 2.08 |
| Bison Bison | | | | | | | | |
| Oligo-1 | | | | | | | | |
| Virus | 4925.01 | 8.99 | 41.9 | 10153.68 | 7.99 | 6.92 | 51.18 | 2.07 |
| Bison Bison | | | | | | | | |
| Oligo-2 | | | | | | | | |

1. A composition comprising; a first agent that binds to a negative-strand ribonucleic acid (RNA) molecule produced by a coronavirus, wherein the negative-strand ribonucleic acid (RNA) molecule comprises a 5′ poly-U stretch, and wherein the first agent is selected from the group consisting of a nucleic acid, an antibody, a protein, an aptamer, and a low molecular weight compound,
wherein the first agent is a nucleic acid molecule comprises a poly-A stretch that is between 5-50 bases in length,
the poly-A stretch is capable of hybridizing to the 5′ poly-U stretch in the negative-strand RNA molecule, and
comprises a 5′-end linker region that comprises a sequence complementary, or nearly complementary, to a sequence at the 5′-end of the negative-strand RNA that starts immediately after the poly-U stretch.

2. (canceled)

3. (canceled)

4. (canceled)

5. The composition of claim 1, wherein the linker sequence in the nucleic acid molecule comprises GGAAGAATCAC (nucleotides 1-11 of SEQ ID NO: 1) or GAATTGGAAGATCAC (nucleotides 1-15 of SEQ ID NO: 2).

6. (canceled)

7. The composition of claim 1, wherein the nucleic acid molecule contains at least one of a nucleic acid analogue, a base analog, a modified base, and an abasic site.

8. The composition of claim 7, wherein the at least one of a nucleic acid analogue, a base analog, a modified base, and an abasic site is the 3′ terminal nucleic acid, the 5′ terminal nucleic acid, or both the 3′ and 5′ terminal nucleic acids.

9. (canceled)

10. (canceled)

11. The composition of claim 8, wherein the 3′ and 5′ terminal nucleic acid modifications are the same.

12. The composition of claim 8, wherein the 3′ and 5′ terminal nucleic acid modifications are different.

13. The composition of claim 7, wherein at least one of the nucleic acid analogues is a 2′MOE ribonucleic acid.

14. (canceled)

15. (canceled)

16. (canceled)

17. (canceled)

18. The composition of claim 1, wherein the composition further comprises a delivery vehicle.

19. The composition of claim 18, wherein the delivery vehicle is a polymer.

20. (canceled)

21. (canceled)

22. (canceled)

23. The composition of claim 1, wherein the virus is from the family Coronaviridae.

24. The composition of claim 23, wherein the virus is MHV or SARS-CoV-2.

25. (canceled)

26. A method for treating a coronavirus infection, comprising administering to a subject the composition according to claim 1.

27. The method of claim 26, wherein the a coronavirus infection is a SARS-CoV-2 infection.

28. The method of claim 26, wherein the a coronavirus is SARS-CoV-2.

29. (canceled)

30. (canceled)

31. (canceled)

32. The method of claim 31, wherein the linker sequence in the nucleic acid molecule comprises GGAAGAATCAC (nucleotides 1-11 of SEQ ID NO: 1) or GAATTGGAAGATCAC (nucleotides 1-15 of SEQ ID NO: 2).

33. (canceled)

34. The method of claim 28, wherein the nucleic acid molecule contains at least one of a nucleic acid analogue, a base analog, a modified base, and an abasic site.

35. The method of claim 34, wherein the at least one of a nucleic acid analogue, a base analog, a modified base, and an abasic site is the 3′ terminal nucleic acid, the 5′ terminal nucleic acid, or both the 3′ and 5′ terminal nucleic acids.

36. (canceled)

37. (canceled)

38. The method of claim 35, wherein the 3′ and 5′ terminal nucleic acid modifications are the same.

39. The method of claim 35, wherein the 3′ and 5′ terminal nucleic acid modifications are different.

40. The method of claim 34, wherein at least one of the nucleic acid analogues is a 2′MOE ribonucleic acid.

41. (canceled)

42. (canceled)

43. (canceled)

44. (canceled)

45. The method of claim 26, wherein the composition further comprises a delivery vehicle.

46. The method of claim 45, wherein the delivery vehicle is a polymer.

47. (canceled)

48. (canceled)

49. (canceled)

50. The method of claim 26, wherein the virus is from the family Coronaviridae.

51. The method of claim 50, wherein the virus is MHV or SARS-CoV-2.

52. (canceled)
